(19) United States
(12) Patent Application Publication
(10) Pub. No.: US 2018/0305757 A1
(43) Pub. Date: Oct. 25, 2018

(54) Repetitive Element (RE)-based Genome Analysis and Dynamic Genetics Surveillance Systems

(71) Applicants: The Regents of the University of California, Oakland, CA (US); Korea Advanced Institute of Science and Technology, Daejeon (KR); Shriners Hospitals for Children, Tampa, FL (US)

(72) Inventors: Kiho Cho, Davis, CA (US); David Greenhalgh, Davis, CA (US); Dong Ho Cho, Daejeon (KR)

(21) Appl. No.: 15/768,410

(22) PCT Filed: Oct. 17, 2016
(86) PCT No.: PCT/US2016/057390

§ 371 Date: Apr. 13, 2018

Related U.S. Application Data
(60) Provisional application No. 62/242,858, filed on Oct. 16, 2015.

(51) Int. Cl. C12Q 1/6881; G06F 19/26; G06F 19/22; G06K 9/00

(52) U.S. Cl. CPC C12Q 1/6881; G06F 19/26; G06K 2209/07; G06K 9/00147; G06F 19/22

(57) ABSTRACT

Methods for determining a genetic identity of a cell, tissue, organ, or organism, based on type, position, and size of every occurrence of at least one repetitive element in the genome of the cell, tissue, organ, or organism. The methods can include using a computer to generate a graphical representation of the genetic identity of the cell, tissue, organ, or organism, and comparing genetic identity at different times/spaces. Also described herein is a computer implemented Universal Genome Information System, which serves as a genome-RE/TRE information management and analysis platform.

### FEDERALLY SPONSORED RESEARCH OR DEVELOPMENT

This invention was made with Government support under Grant No. GM071360 awarded by the National Institutes of Health. The Government has certain rights in the invention.

### TECHNICAL FIELD

Described are methods for determining a genetic identity of a cell, tissue, organ, or organism, based on type, position, and size of every occurrence of at least one repetitive element in the genome of the cell, tissue, organ, or organism. The methods can include using a computer to generate a graphical representation of the genetic identity of the cell, tissue, organ, or organism, and comparing genetic identity at different times/spaces. Also described herein is a computer implemented Universal Genome Information System, which serves as a genome-RE/TRE information management and analysis platform.

### BACKGROUND

The vast majority of core concepts and relevant methodologies for modern studies of both normal and disease biology are stringently tethered to the function and polymorphism of “conventional” genes. Conventional gene sequences are reported to be shared, with a homology of greater than 80%, among a wide range of species, ranging from rodents to humans (Consortium, 2002; Guenet, 2005). A careful examination of the data obtained from recent biomedical investigations, which focus on the function and polymorphism of conventional genes, indicates that the ratio of tangible and/or helpful returns is very low, in consideration of the enormous investments (Padyukov, 2013; Seok et al., 2013; Shastry, 2002; Takao and Miyakawa, 2015a; Takao and Miyakawa, 2015b).

The announcements in 2001 and 2002 that the human and mouse reference genomes, respectively, were “completely” sequenced was followed by numerous publications which reported “whole” genome sequences of a wide range of species (Consortium, 2004; Consortium, 2002; Herrero-Medrano et al., 2014; Jun et al., 2014; Lander et al., 2001; Mullikin et al., 2010; Venter et al., 2001). These whole genome projects were executed apparently on a platform of a static genome within an individual (Fujimoto et al., 2010; Kim et al., 2009; Zhang et al., 2014). However, it is interesting to note that the human and mouse reference genomes have not been fully decoded as of May 2015 (National Center for Biotechnology Information [NCBI], National Institutes of Health). For instance, less than half of the human chromosome Y has been decoded.

### SUMMARY

It is estimated that the sum of all conventional gene sequences (exons) represents less than ˜1.2% of the human and mouse genomes, which have not been completely sequenced yet. Currently, genetics surveillance protocols for humans, animals, and plants primarily focus on polymorphisms in small sets of conventional gene and/or microsatellite sequences. The limited information obtained from conventional gene and/or microsatellite polymorphism analyses is apparently inadequate for precise genetic surveillance/identification. In fact, the results from the present studies, in which our novel Repetitive Element (RE; or repetitive genetic element)-based genome-landscaping technologies were tested with genomic DNAs from different humans and mouse strains, demonstrated that the current conventional gene/microsatellite-based protocols provide insufficient data for the correct identification of individual genomes. Described herein are REome and Transposable Repetitive Element-ome (TREome) analysis-based systems and methods that enable high-resolution and tunable genetics surveillance/identification systems for both static and dynamic (temporal and spatial) genomes of all life forms. These genetics surveillance/identification systems are applicable to a wide range of species (e.g., humans, animals, plants, and microbes) and fields, such as justice forensics, animal breeding, plant breeding, pharmacogenomics, monitoring of radiation therapy, cell/tissue typing, diagnostics-marker discovery, fundamental cell biology and genetics.

Thus, in a first aspect, the invention provides methods of determining a genetic identity for a cell or organism. The methods can include determining type, position, and size of every occurrence of at least one repetitive element (both transposable and non-transposable) in the genome of the cell or organism; thereby determining the genetic identify of the cell or organism.

A computer-implemented method of generating a graphical representation, e.g., an RE array, of the genetic identity of a cell or organism. The methods include receiving electronic information regarding the type, position, and size of every occurrence of at least one repetitive element in the genome of the cell or organism; and using a processor to generate a graphical representation of the electronic information, e.g., by self-alignment of a query sequence to determine direct (illustrated by blue angles on FIG. 19) REs and inverse (illustrated by red angles on FIG. 19) REs followed by dot-matrix presentation of their occurrences and positions. RE arrays are preferably formed by combinatorial organization of occurrences and positions of direct and inverse RE sets.

In some embodiments, the cell is from an animal, e.g., a mammal, bird, fish, or reptile; plant; fungus; or bacterium.

In some embodiments, the assay comprises using PCR and/or inverse-PCR (I-PCR) to determine position and sequencing to determine type, size, and/or copy number.

In some embodiments, the electronic information was obtained using PCR and/or inverse-PCR (I-PCR) to determine position and sequencing to determine type, size, and/or copy number.

In some embodiments, the repetitive element is a Transposable Repetitive Element (TRE). In some embodiments, the TRE is an endogenous retrovirus (ERV), long interspersed nuclear element (LINE), short interspersed nuclear element (SINE), or DNA transposon. In some embodiments, wherein the repetitive element is a non-transposable repetitive element.

In some embodiments, the type is based on primary sequence; the position is relative to a reference genome; and/or the size refers to the length or number of repeats.

In some embodiments, using a processor to generate a graphical representation of the electronic information comprises unbiased self-alignment and dot-matrix plot visualization.

In some embodiments, the method includes displaying the graphical representation (e.g., RE array) electronically on a display device to provide a visible image.

In some embodiments, the genetic identity is determined at a specific time or space.

In some embodiments, the genetic identity is determined at a first time or space, and the method further comprising determining genetic identity at a second time or space, and comparing the genetic identity at the first and second time or space to detect changes in the genetic identity of the cell or organism. For example, the methods can be used to monitor change of state (e.g., progression of disease or temporal surveillance) or to identify risk factors or prognostic applications.

In some embodiments, the second time is later than the first time.

In some embodiments, the second space is obtained from a different cell, tissue, or organ within the same organism.

Also provided herein is a computer-implemented method for determining genetic identity of a cell, tissue, organ, or organism, comprising:

accessing, by one or more processing devices, a database (e.g., a Genome Information System as described herein) to obtain data elements comprising genomic sequence information, gene information, genetic variation information, and repetitive element information for a cell, tissue, organ, or organism at a selected time and/or space; computing a genetic identity for the cell, tissue, organ, or organism at the selected time and/or space, wherein the genetic identity is computed based on the data elements; and storing, at a storage location, a representation of the genetic identity.

Also provided herein is a computer-implemented method, comprising:

accessing, by one or more processing devices, a database (e.g., a Genome Information System as described herein) to obtain data elements comprising genomic sequence information, gene information, genetic variation information, and repetitive element information for a cell, tissue, organ, or organism at a selected time and/or space;

obtaining additional information relating to genomic sequence information, gene information, genetic variation information, and repetitive element information in the cell, tissue, organ, or organism, wherein the additional information is associated with a predetermined time and/or space, e.g., aging, stress, and/or disease; and updating the data elements.

In some embodiments, the methods include computing a genetic identity for the cell, tissue, organ, or organism, wherein the genetic identity is computed based on the data elements; and

storing, at a storage location, a representation of the genetic identity.

Also provided herein is a computer-implemented system (e.g., a Genome Information System as described herein) for storing genomic information, comprising: memory storing computer-readable instructions,

one or more processing devices configured to execute the computer-readable instructions to perform operations comprising:
accessing a database to obtain data elements comprising genomic sequence information, gene information, genetic variation information, and repetitive element information for a cell, tissue, organ, or organism at a selected time and/or space;
computing a genetic identity for the cell, tissue, organ, or organism at the selected time and/or space, wherein the genetic identity is computed based on the data elements; and storing, at a storage location, a representation of the genetic identity.

In some embodiments, the selected time and/or space is different from the predetermined time and/or space; e.g., they can be the first and second time/space as described herein (in either order). In some embodiments, the selected and predetermined time/space are the same.

In some embodiments, the representation of the genetic identity is usable for generating an image of the genetic identity.

In some embodiments, the method includes presenting the image of the genetic identity on a display device.

In some embodiments, the selected time and/or space relates to changes associated with aging, stress, and/or disease.

The present disclosure also provides methods of determining origin of a test subject. The methods include the steps of determining type, position, and size of every occurrence of at least one repetitive element in the genome of the test subject; comparing the type, position, and size of every occurrence of the repetitive element of the test subject to the type, position, and size of every occurrence of the repetitive element of a reference subject; determining that the type, position, and size of every occurrence of the repetitive element of the test subject and the type, position, and size of every occurrence of the repetitive element of the reference subject is not statistically different; and identifying the test subject as having the same origin as the reference subject. In some embodiments, the test subject is a human, a plant, or an animal.

In one aspect, the disclosure relates to methods of sub-classifying a disease of humans, plants, and animals. The methods include the steps of determining type, position, and size of every occurrence of at least one repetitive element in the genome of a group of subjects with a disease; applying a clustering algorithm to the type, position, and size of every occurrence of the repetitive element in the genome of the group of subjects; and identifying a sub-group of subjects as having a sub-group disease.

In another aspect, the disclosure relates to methods of determining whether a test cell belongs to a reference cell line. The methods include the steps of determining type, position, and size of every occurrence of at least one repetitive element in the genome of the test cell; comparing type, position, and size of every occurrence of the repetitive element in the genome of the test cell to type, position, and size of every occurrence of the repetitive element in the genome of a reference cell from the reference cell line; determining that the type, position, and size of every occurrence of the repetitive element of the test cell is not statistically different from the type, position, and size of every occurrence of the repetitive element of the reference cell; and identifying the cell as belonging to the cell line.

The present disclosure also provides methods of identifying a locus associated with a disease. The methods include the steps of determining type, position, and size of every occurrence of at least one repetitive element in the genome of a first sibling with the disease; comparing type, position, and size of every occurrence of the repetitive element in the genome of the first sibling to type, position, and size of every occurrence of the repetitive element in the genome of a second sibling, wherein the second sibling does not have the disease; and identifying the locus associated with the disease. In some embodiments, the first sibling and the second sibling are of the same sex.

As used herein, the term “significant” or “significantly” refers to statistical significance (or a statistically significant result) is attained when a p-value is less than the significance level (denoted a, alpha). The p-value is the probability of obtaining at least as extreme results given that the null hypothesis is true whereas the significance level a is the probability of rejecting the null hypothesis given that it is true. In some embodiments, the significance level is 0.05, 0.01, 0.005, 0.001, 0.0001, or 0.00001, etc. In some embodiments, “significantly different” refers to the difference between the two groups have attained the statistical significance.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIGS. 1A-B. Variations in Transposable Repetitive Element (TRE)-ome landscapes among the germ-line genomic DNAs isolated from sperm of C57BL/6J inbred mice of different age groups. A. Illustration of inverse-PCR (I-PCR) strategy for TREome landscaping. The processes of I-PCR for TREome landscaping analyses of genomic DNAs are illustrated. E (restriction enzyme recognition site); MLV-ERV (murine leukemia virus-type ERV); P (primer); Ref. (reference); ISL-F1 and ISL-R1 (primer names). B. Variations in MLU-ERV-probed TREome landscapes among the germ-line genomic DNA samples of C57BL/6J inbred mice. There are visible variations in the TREome landscapes, which are reflected in I-PCR amplicon banding patterns, among the genomic DNAs isolated from the sperm of three age groups (8-, 12-, and 20-weeks) of C57BL/6J inbred mice. Each lane represents the genomic DNA of sperm samples from a single mouse. Wk (week); SM (size marker)

FIGS. 2A-B. Spatial variations in the TREome landscapes among germ-line and somatic genomes from a single C57BL/6J inbred mouse. A. Variable TREome landscapes of 13 non-lymphoid organ and sperm genomic DNA samples. TREome landscapes were highly variable among the 13 non-lymphoid organ and sperm genomic DNA samples derived from a single C57BL/6J mouse although they share a collection of I-PCR amplicon bands. SM (size marker); SP (sperm); AG (adrenal gland); BR (brain); CH (cerebellar hemisphere); HE (heart); KI (kidney); LI (liver); LU (lung); PA (pancreas); SG (salivary gland); SI (small intestine); SK (skin); SV (seminal vesicle); TE (testes). B. Variable TREome landscapes of five lymphoid organ and sperm genomic DNA. Five lymphoid organ and sperm genomic DNA samples, derived from the same mouse as in panel A, had variations in banding patterns of I-PCR amplicons. SM (size marker); SP (sperm); BM (bone marrow); TH (thymus); SP (spleen); M-LN (mesenteric lymph node); I-LN (inguinal lymph node).

FIG. 3. Variable TREome landscapes in different brain compartments of C57BL/6J inbred mice. Variations in the I-PCR amplicon banding patterns were visible among the six different brain compartments of two 5-week old female C57BL/6J mice. In addition, within each compartment, the landscape patterns were not shared by the two mice. SM (size marker); BS (brain stem); CB (cerebral cortex); CC (corpus callosum); CH (cerebellar hemisphere); HI (hippocampus); OF (olfactory bulb); KI (kidney).

FIG. 4. Variations in TREome landscapes in the immune organs (primary-thymus and secondary-spleen) of C57BL/6J inbred mice of different ages. Substantial variations in the TREome landscapes were observed in the thymus and spleen genomic DNA among all four age groups (5-, 8-, 12-, and 20-weeks) of C57BL/6J male mice. No age-specific or immune organ-specific I-PCR amplicon banding patterns were identified. Wk (week); SM (size marker).

FIG. 5. Polymorphic TREome landscapes of spatially separated organ sets of a C57BL/6J mouse. There were considerable variations in TREome landscapes among the individual sets of three lymph nodes (thoracic mammary, inguinal mammary, and mesenteric) and two mammary fat pads (#2-right and #4-right) derived from a 27-month old female C57BL/6J mouse. In contrast, bone marrow samples isolated from left and right femurs shared I-PCR amplicon patterns which are visibly different (density and banding pattern) from the landscapes of lymph nodes and mammary fat pads. SM (size marker); TM-LN (thoracic mammary lymph node); IM-LN (inguinal mammary lymph node); FP (fat pad); L (left); R (right); BM (bone marrow).

FIGS. 6A-B. Variable TREome landscapes in C57BL/6J inbred mice depending on gender and individual. A. Gender-specific TREome landscapes. There were apparent variations in TREome landscapes of the kidney and liver genomes between females and males of 19-month old C57Bl/6J mice; these variations include one distinct I-PCR amplicon band (indicated with an arrow) found only in male mice in the kidney and liver genomes. In addition, TREome landscapes of the liver genomes lack a set of amplicon bands in contrast to the kidney genomes (*). Furthermore, each mouse (female or male) had either one of the two distinct sets of I-PCR amplicon bands, regardless of gender (**). SM (size marker); KI (kidney); LI (liver); F (female); M (male). B. Differences in copy number of a TREome family (MLU-ERVs) between females and males. There were higher copy numbers of MLV-ERVs in the kidney and liver genomes of the same male mice as described in panel A in comparison to female mice. Interestingly, the kidney genomes had more MLU-ERV copies compared to the liver genomes in all three male mice, but not in females.

FIGS. 7A-B. High-level diversity in MLU-ERV (TREome) LTR sequences among 12 laboratory mouse strains. The extent of variations among the population of MLU-ERV LTR sequences from the genomic DNA of 12 mouse strains were examined by a probability distribution function analysis for 4-nucleotide word sets. The extent of variations for the individual words were visualized on a 16×16 (=256) matrix. In contrast to the overall low variation matrix of GAPDH genes (a conventional gene) from 20 laboratory mouse strains (B), the vast majority of the words from the MLV-ERV sequences derived from 12 mouse strains (A) had a high-level variability. Variability of each word within individual sets (12 [MLU-ERV LTR] strains or 20 [GAPDH gene] strains) is coded on a gray scale, ranging from white (low=0) to black (high=0.001261). Note that FIGS. 7A and 8-13 used primers in Table 1; FIG. 7B used GAPDH primer in Table 4.

FIG. 8. Polymorphic TREome landscapes among the genomes of 56 laboratory mouse strains. Using MLV-ERVs as a probe, TREome landscapes of the genomes of 56 laboratory mouse strains were visualized. None of the 56 mouse strains share the same TREome landscape patterns. Distinct TREome landscape patterns were found within certain families of strains (highlighted with dotted lines and colors), such as 129PI-XI/, A/, BALB/, C3H/, C57BL/, DBA/, and MOL. The TREome landscapes of the C3H/HeJ and C3H/HeOuJ strains were different (one of the different bands is indicated with an arrow). In addition, three strains (Mus caroli/Ei, Mus Pahari/Ei, and PANCEVO/Ei) had only a couple of visible bands. SM (size marker)

FIG. 9. Un-identical TREome landscapes between the C3H/HeJ strain and its wildtype control, C3H/HeOuJ strain. With respect to the TREome landscape patterns, the C3H/HeJ strain (TLR4−/−) is markedly different from its presumed wildtype control, C3H/HeOuJ (TLR4+/+) strain in the genomes of all six organs (kidney [KI], liver [LI], lung [LU], lymph node [LN], spleen [SP], and thymus [TH]) examined. In particular, one distinct TREome band (enlarged in a separate window) was found only in the C3H/HeJ strain while another band was present only in the C3H/HeOuJ strain. SM (size marker)

FIG. 10. Apparent variations in TREome landscapes between the CD14 knock-out strain and its backcross-control, C57BL/6J strain. There were apparent differences in the TREome landscapes between the genomes of CD14 knock-out and its backcross-control (C57BL/6J) strains in all six organs (kidney [KI], liver [LI], lung [LU], lymph node [LN], spleen [SP], and thymus [TH]) examined. Two unique TREome bands were visible only in the CD14 knock-out strain, whereas two other bands were found only in the C57BL/6J control strain. SM (size marker)

FIG. 11. TREome landscapes of 129 mouse substrains. Genomic DNAs of nine 129 substrains (129S1/SvImJ [A], 129/Sv-Lyntm1Sor/J [B], 129S1/Sv-Oca2+ Tyr+ Kit1S1-J/J [C], 12954/SvJae-Inhbbtm1Jae/J [D], 12954/SvJae-Pparatm1Gonza [E], 12954/SvJaeSor-Gt(ROSA)26Sortm1(FLP1)Dyma [F], 12956/SvEv-Mostm1Ev/J [G], 129P1/ReJ [H], and 129X1/SvJ [I]) were examined for variations in TREome landscapes. Overall, all substrains shared a similar TREome landscape pattern; however, both of the 129/Sv-Lyntm1Sor/J and the 129P1/ReJ substrains lacked a unique band in its TREome landscape compared to the other seven strains. SM (size marker)

FIG. 12. TREome landscape-based monitoring of genome-crossing between two mouse strains: an example. We examined whether the genome-crossing events between two mouse strains (C57BL/6J×129S1/Sv1mJ) are reflected in the TREome landscapes of a hybrid offspring. For the most part, the pattern of the F2 hybrid's TREome landscape included the bands from both the C57BL/6J and 129S1/Sv1mJ genomes; however, certain bands specific for the individual parental strains were not present. This F2 hybrid's TREome banding pattern provides valuable information which visualizes the genomic status of crossing events between two strains. SM (size marker)

FIGS. 13A-B. Highly polymorphic TREome gene (MMTV-ERV SAg) isoforms among 46 laboratory mouse strains. Polymorphisms in the MMTV-ERV SAg gene coding regions among 46 laboratory mouse strains were examined. A. A high-level of MMTV-ERV SAg gene polymorphism was apparent among the genomes of the 46 mouse strains; altogether, 183 MMTV-ERV SAg gene isoforms were identified. In addition, the C-terminus regions of the MMTV-ERV SAg gene coding sequences had relatively high levels of variations compared to the other regions. B. MMTV-ERV SAg isoform profiles were different between the C3H/HeJ inbred strain (eight unique isoforms) and its wildtype TLR4 control strain, C3H/HeOuJ (five unique isoforms).

FIGS. 14A-B. (A) Percentages of transposable and nontransposable elements in the human genome. (B) Diagram showing the possible shared and unique TREs in the genomes of different individuals.

FIG. 15. The core platforms of the Dynamic Genetics Surveillance Systems (DGSS) versus the current surveillance systems employed in research and diagnostics market. The current genetics identification/surveillance systems primarily focus on the function and polymorphism of conventional genes on a static genome platform. In contrast, the DGSS interrogates inherent diversity and acquired activity of TREs on a dynamic genome platform in conjunction with conventional genes to compute precision genetics surveillance/diagnostics values. RE (repetitive element); TRE (transposable RE).

FIG. 16A is a histologic image showing different pathologic areas analyzed from a breast biopsy (less than 20 mm in length) taken from a subject diagnosed with breast cancer. TDLU, terminal ductal lobular unit (normal); DCIS, ductal carcinoma in situ (non-invasive early cancer)

FIG. 16B is a pair of images of TREome landscapes, which were resolved by polyacrylamide gel electrophoresis, using two different HERV (human endogenous retrovirus) family sequences as landscaping probes. It is interesting to note that TREome landscapes are highly divergent even within a small biopsy sample from a single patient. It is possible that the TREome landscape patterns are pathologic status-specific; thus, examination of TREome landscape patterns may reveal prognostic/diagnostic signatures, such as DCIS-specific TRE landscapes, in contrast to malignant breast tumor-specific TRE landscapes.

FIG. 17 is an image of a polyacrylamide gel showing TREome landscapes represented by HERV amplicons (HERV type and position) in white blood cells collected from a subject at 16 time points after a burn injury. Temporal changes in TREome landscapes in the white blood cell genomes of a single burn patient are evident.

FIG. 18 is a pair of images showing the results of TREome landscape analysis using murine endogenous retrovirus sequences as probes in brain and skin of C57BL/6 inbred mice at 2 weeks, 29 weeks, and 77 weeks of age. The genomic TREome landscapes change markedly as mice age. In addition, within a single mouse, the landscapes vary depending on tissue type.

FIG. 19 is an exemplary schematic illustrating the generation of a graphical representation of a repeat element array. To identify RE arrays, self-alignment of a query sequence was performed to mine direct (blue angle) REs and inverse (red angle) REs followed by dot-matrix presentation of their occurrences and positions. RE arrays are formed by combinatorial organization of occurrences and positions of direct and inverse RE sets.

FIGS. 20A and B are sets of exemplary RE arrays from human (A) and mouse (B) showing that the patterns are unique for each species, in contrast to ˜85% sequence homology for conventional genes. These findings suggest that RE arrays contribute to phenotypic details unique for each species.

FIG. 21 shows exemplary comparison between the NCBI reference RE array with locus-matching arrays from individuals of Chinese or Korean ancestry. Although the overall configuration of the locus-matching three arrays are almost identical, a close examination identifies multiple configurational polymorphisms unique for the individual arrays of different ancestry.

FIG. 22A shows three different PCR strategies for structural analyses of the central tandem repeat cluster within the RE arrayCHR7.32 locus on chromosome 7. The tandem repeat cluster (in both forms of dot-matrix structure and linear-visualization of mosaic repeat units) within the RE arrayCHR7.32 locus is outlined with red lines. PCR primers for three different types of structural analyses of the highlighted tandem repeat cluster are mapped: 1) staggered PCR (A/B/C/D amplicons: NF1-1A, NF5-1A, NF3-2A, NF5-2A, and NF6-2A), 2) repeat unit-length PCR (UL amplicon: Obox4-1B and NF8r-2B), and 3) I-PCR (IN amplicon: DiT-1B and DiT-2D). In addition, putative PCR amplicon sizes expected from the individual primer sets without any recombination events are provided as a reference.

FIG. 22B shows structural variations in the central tandem repeat cluster within the RE arrayCHR7.32 locus. Six organs/tissues from five age groups of C57BL/6J female mice were surveyed for structural variations in the tandem repeat cluster within the RE arrayCHR7.32 locus using staggered PCR primer sets. Organ/tissue-specific and age-dependent structural variations in the tandem repeat cluster within the RE arrayCHR7.32 locus were identified in the five age groups (0 week [neonate], 2 weeks, 6 weeks, 12 weeks, and 29 weeks) by examining four sets of the PCR amplicons (A, B, C, and D) which are generated using staggered primer pairs (FIG. 22A). wk (week); G (GAPDH).

FIG. 23 is a schematic illustration of an exemplary Genome Information System (GIS). Conventional genes have multiple short and long distance relationships forming a network of interacting blocks of information. While the exome of conventional genes (yellow stars) consists of only ˜1.2% of this patchwork, the functions of the other ˜98% of the genome are largely unaccounted for (left). Once the functions of both the exome and non-exome regions (e.g., TREs, TRE genes, micro RNAs) are cataloged, a more distinctive GIS pattern will emerge (middle). The addition of dynamic (temporal and spatial) changes, associated with aging, stress, and disease, will define a more comprehensive pattern identifying the critical targets for precision biology and medicine (right).

FIG. 24 is a schematic illustration of an exemplary Universal Genome Information System, which serves as the genome-RE/TRE information management and analysis platform. Although the figure refers to TREs and TREgenes, any REs can be included.

### DETAILED DESCRIPTION

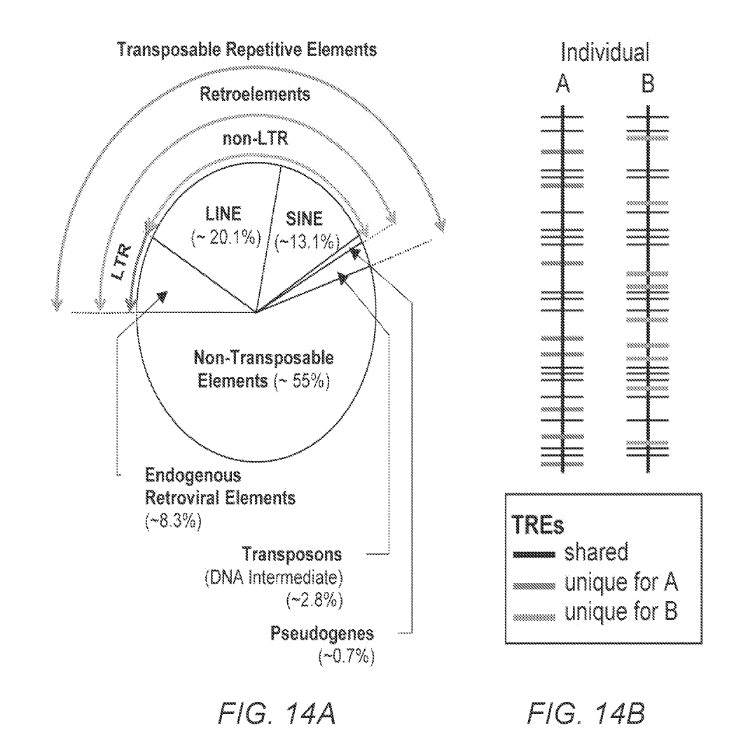

Transposable repetitive elements (TREs) make up about 45% of the human genome (FIG. 14A) and are present in all vertebrates examined so far. Individuals may share certain transposable elements within their genome. However, our recent studies involving different mouse strains and human subjects suggest there are also significant polymorphisms in the TREs, in particular the endogenous retroviruses (ERVs), that may constitute a unique profile for each person (FIG. 14B). According to the current annotation information from major human and mouse genome databases, the sum of exons pertaining to the conventional genes is estimated to occupy ˜1.2% of the full-length genomes (Consortium, 2002; Lander et al., 2001). On the other hand, repetitive elements, named the “REome” herein, and transposable repetitive elements, named the “TREome” herein, are expected to make up about 46% and ˜38.6% of the human and mouse genomes, respectively (Consortium, 2002). There are four main families of the TREome: endogenous retroviruses (ERVs), long interspersed nuclear elements (LINEs), short interspersed nuclear elements (SINEs), and DNA transposons. Often, the transcription products of ERVs are referred as “non-coding” long RNA species; however, it has been documented over last a few decades that some ERVs (mostly mouse, pig, and human) are coded with “unconventional” genes capable of producing proteins of specific function (Bittmann et al., 2012; Lee et al., 2011; Nakagawa and Harrison, 1996). In addition, temporally and spatially acquired activities of the REome/TREome are capable of altering the genome configuration by “copy and paste” function (ERVs, LINEs, and SINEs) (Bohne et al., 2008; Parisod et al., 2010).

Publications and databases commonly define the sizes of the genomes and/or chromosomes of various species, such as human and mouse reference genomes at NCBI, based on an assumption that their configurations are fully characterized and rather static (Church et al., 2015; Rosenfeld et al., 2012). The finding that the size of NCBI's build Annotation Release 105 reference mouse chromosome Y of ˜92 Mb in size is almost six times larger than the build 37.2's ˜16 Mb indicates that the current size estimates of genomes and/or chromosomes of various species need to be reevaluated (Lee et al., 2013). A recent report that the size and structure of the C57BL/6J inbred mouse genomes are temporally and spatially changed in conjunction with differential TREome activities suggests that: 1) it is impractical to identify a single representative genome for an individual mouse or human from a pool of variant genomes and 2) genome dynamicity is linked to a range of biological processes, such as differentiation, stress response, and aging (Lee et al., 2012; Lee et al., 2015).

It is unlikely that the limited library of proteins derived from the current repertoire of conventional genes, in conjunction with “non-coding” RNA species, is sufficient to explain the enormous extent of phenotypic polymorphisms observed in both normal and disease states. Thus far, the majority of the efforts for understanding a host of normal and disease phenotypes, based solely on the knowledge derived from studies of the function and polymorphism of conventional genes, have been inefficient. As a new functional layer of the genome that contributes to the development of disparate normal and disease phenotypes in humans and other species, we introduce the inherent diversity and acquired activity of repetitive elements, or REs, and transposable repetitive elements, or TREs. In contrast to the common polymorphisms of conventional genes observed in a population, variations in the genomic RE/TRE landscapes should be directly linked to the differential shaping of the genomes of somatic cells within an individual. The Universal Genome Information System (described below) can help dynamically manage the TREome as well as other genomic data and introduce new insights into the variable, often individual-specific, biological mechanisms (both normal and disease) and identify novel RE/TRE loci as markers for specific phenotypes.

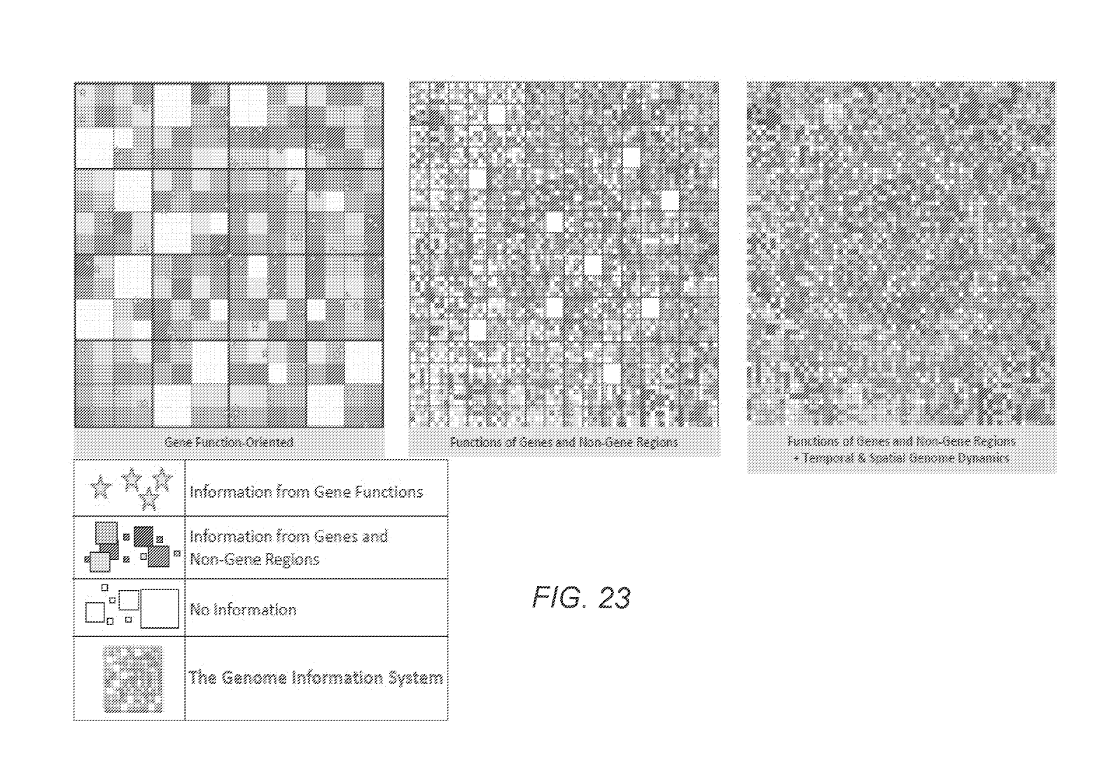

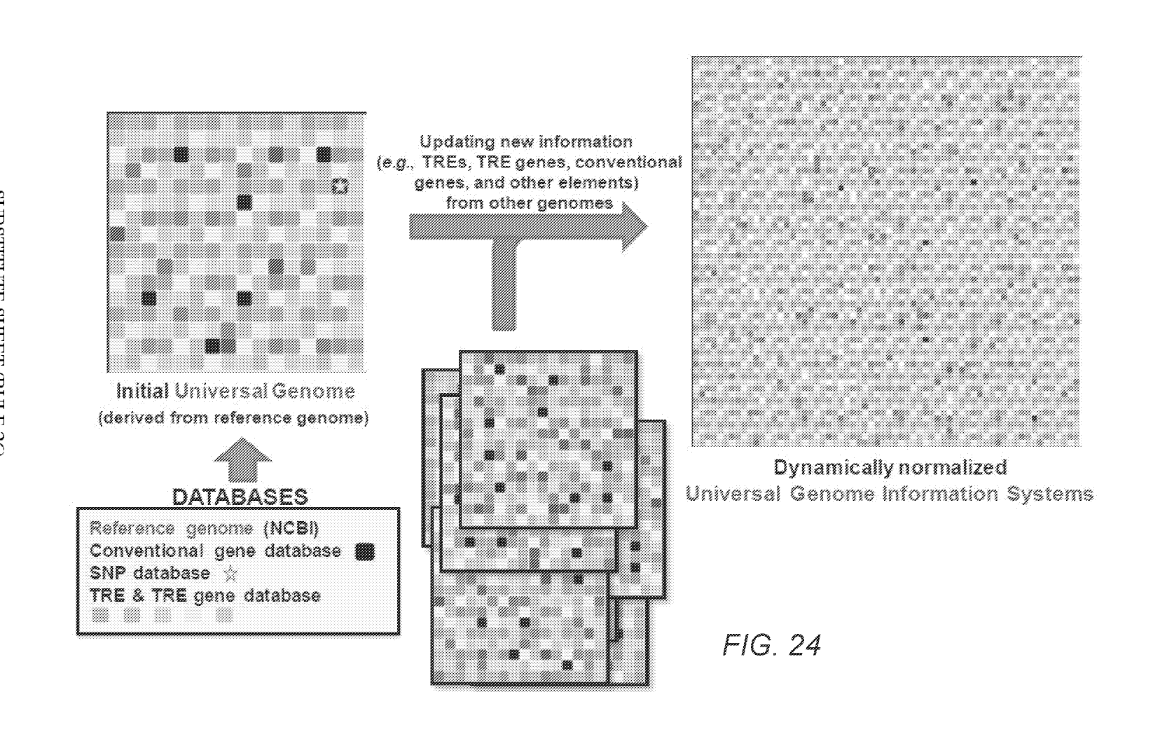

As described herein, it would be logical to explain aging-related phenotypic changes in the context of a dynamic genome rather than a static one. From the perspective of precision biology and medicine, some of the normal and disease phenotypes, which have been explained by functions of conventional genes, need to be re-evaluated to account for the effects of both the inherent diversity and acquired activity of the RE/TREs and the associated dynamic nature of the genome. It is certain that normal and disease phenotypes, which sequentially or randomly appear during the lifetime of an individual, are not statically forged by only the standard exome (from conventional genes), but by the entire genome and its innate temporal and spatial dynamics. This method of collecting and interpreting data from the entire genome information system enables precision decoding of biology and medicine; see, e.g., FIG. 23, which illustrates an exemplary Universal Genome Information System (GIS). Conventional genes have multiple short and long distance relationships forming a network of interacting blocks of information. While the exome of conventional genes (yellow stars, right panel of FIG. 23) consists of only ˜1.2% of this patchwork, the functions of the other ˜98% of the genome are largely unaccounted for (FIG. 23, left). Once the functions of both the exome and non-exome regions (e.g., TREs, TRE genes, micro RNAs) are cataloged, a more distinctive GIS pattern will emerge (FIG. 23, middle). The addition of dynamic (temporal and spatial) changes, associated with aging, stress, and disease, will define a more comprehensive pattern identifying the critical targets for precision biology and medicine (FIG. 23, right).

Genetics Surveillance System (GSS)

Currently, both normal and disease biology is explained in the context of the function and polymorphism of conventional genes. However, it is clear that certain phenotypes cannot be explained solely on the basis of conventional gene functions. For example, the genomes of mice and humans share a high degree of homology (˜85%) in their standard exome sequences (conventional genes). Hence, the evident phenotypic differences between these species cannot be explained by exome functions alone. The standard exome comprises only ˜1.2% of our genome, and the vast majority of the residual genome is occupied by a plethora of repetitive elements (REs), often called “Junk” DNA. In particular, transposable repetitive elements (TREs), constituting at least 45% of the human genome, have the potential to dynamically shape the genomes' configuration through “copy and paste” and “cut and paste” functions. There are a myriad of heterologous TRE families in the human and mouse genomes, which include endogenous retroviruses (ERVs), long interspersed nuclear elements (LINEs), short interspersed nuclear elements (SINEs), DNA transposons, and unknowns.

We demonstrated that human and mouse REs, e.g., TREs (Human ERVs [HERVs] and mouse ERVs), are inherently diverse and that injury-elicited stressors activate certain HERVs in an individual- and disease course-specific manner. The expression of injury-associated HERV polypeptides differentially induced inflammatory mediators (e.g., IL-6, IL-1β). In addition, the genomic landscapes of hypertrophic burn scars, in the context of type and position of HERVs, were altered compared to the matching control skin, suggesting HERVs' roles in hypertrophic scar development. Our recent studies further showed that the genomic TRE/ERV landscapes are altered in various human and mouse tumors in comparison to their matching controls (unpublished). In particular, following micro-dissection of a paraffin section from a patient's histological normal, precancer, and cancer breast biopsy, it was determined that each of the regions has a unique genomic landscape of TREs/HERVs.

Described herein is a high-resolution genetics surveillance system (GSS) that uses the platform of REome/TREome landscapes and TREome genes in conjunction with current conventional gene-based monitoring systems. For example, genetic uniformity of established laboratory mouse strains, both conventional and genetically engineered, could be evaluated by the high-resolution GSS for initial confirmation and maintenance of each strain. Depending on the GSS data collected, additional breeding designs and/or surveillance protocols can be implemented to obtain acceptable levels of genetic uniformity for each strain. When new mouse strains are developed, the GSS serves as a high-resolution tool for dynamic monitoring of genetic crossing/backcrossing. The GSS is applicable across species and organisms (e.g., humans, animals, plants, as well as cells therefrom) and fields (e.g., agriculture, forensics). Identifying and accounting for variations in the REome/TREome landscapes and TREome genes can be used to establish a genetically uniform laboratory mouse strain as well as decoding normal and disease biology in numerous organisms.

The genome information is expected to be variable depending on each individual organism, organ, cell type, stress environment, and age, resulting in non-uniform genome sizes unique for the individual genomic DNA sources. For efficient storage, normalization, and computation of the information residing in these non-uniform and dynamic (temporal and spatial) genomes, a novel genome management system, named the “Universal Genome Information System”, can be used. FIG. 23 illustrates an exemplary Universal Genome Information System, which serves as a genome-RE/TRE information management and analysis platform.

Although the Universal Genome Information System is applicable to the genomes of all living organisms, an exemplary design and construction is described herein based on mouse and human genomes. The Universal Genome Information System has the following three key characteristics: 1) designed to accommodate and manage the critical variations in genomes' structure, sequence, and size due to inherent diversity and acquired activity of REs/TREs depending on organ, cell type, stress environment, and age, 2) able to handle all genome variations (structure, sequence, and size) and they are expandable to annotate newly obtained genetic information (e.g., REs, TREs, TRE genes, conventional genes) to their normalized relative positions, and 3) permitting efficient annotation and rapid analyses of conventional genes, TREs, and other genetic elements residing on the genomes of variable structures and sizes since the positions of the individual elements derived from different genomes are normalized into a single dynamically synchronized and universal frame.

The information in the NCBI's human genome database indicates that the reference genome is incomplete and estimates the complete length to be approximately 3 Gb. Initially, the REs, TREs, TRE genes, conventional genes, and other information, which are obtained from the reference genome and/or RE/TRE databases, can be assembled into the dynamic Universal Genome Information System scaffold. The nucleotide position information of the NCBI's human reference genome will serve as the founding frame for the human Universal Genome Information System. Similar information from additional well-established human genome databases, either fully or partially assembled, will be compared to the NCBI's reference genome frame. Any new information (e.g., nucleotide insertions/deletions-related changes in positions as well as single nucleotide polymorphisms, REs, TREs, TRE genes, conventional genes) can be updated to the original frame so that the nucleotide sequence and position information from all available human genome resources are represented in the human Universal Genome Information System. Thus, the genetic information (e.g., REs, TREs, TRE genes, conventional genes), which are annotated in the individual human genomes of different structure, composition, and size, can be consolidated and normalized into the dynamically synchronized frame of the human Universal Genome Information System, enabling efficient storage, management, and comparison/computation analyses of REs, TREs, conventional genes, and other genetic elements. For instance, alignment analyses of multiple whole chromosomes, which contain a plethora of conventional genes and various RE/TRE families, can be accomplished with minimum computation time since all the nucleotide positions are normalized within one synchronized-normalized frame. This Universal Genome platform can be applied to the genomes of any species. The platform for the Universal Genome Information System can be built with standardized and open-source software as much as possible to leverage the existing advancements in the field.

Dynamic Genetics Surveillance Systems (DGSS)

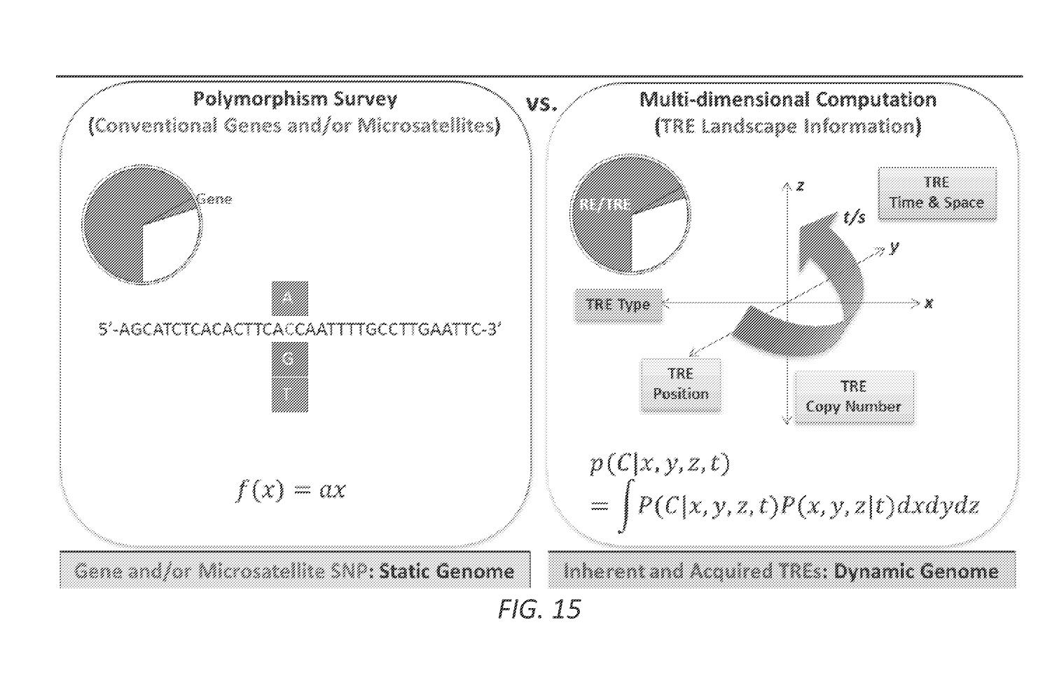

The DGSS produce genetics surveillance data by interrogating the individual genomes for information regarding the RE/TREs' inherent diversity and acquired activity (FIG. 15). For each genome of interest, the multi-dimensional RE/TRE landscape information, with regard to RE/TREs' type, copy number, position, and optionally time/space, is collected using a set of specifically-designed probes by applying a series of DNA-processing protocols, such as PCR, restriction digestion, polyacrylamide gel electrophoresis, capillary electrophoresis, and/or next generation sequencing, and combinations thereof. In some embodiments, inverse PCR (I-PCR) is used. In some embodiments, staggered PCR or repeat unit-length PCR is used. In some embodiments, the sequences of the amplicons derived from the I-PCR and repeat unit-length PCR can be aligned against a reference sequence (e.g., NCBI-based reference genome sequence) to identify shared as well as unshared/deleted regions.

The multi-dimensional RE/TRE landscape data for each genome can be collected and recorded, using methods known in the art, e.g., by: 1) TRE amplicon banding pattern on a polyacrylamide gel or an electropherogram and 2) annotated multi-dimensional information of REs and/or TREs, with regard to their type, copy number, position, and time/space, on a “Universal Genome” scaffold, which can be designed and developed specifically for each species/individual; these can be represented as the RE arrays described herein. TRE/RE copy number and position information for each type of TRE/RE will depend on the specific primer sets used. Comparisons between samples, e.g., samples that differ in time and/or space, can be done for each single primer set or specific combinatorial primer sets. The results from one primer set can be confirmed by the data obtained from another set.

Following sequence analysis, the TRE amplicon-band information can also be annotated within the Universal Genome scaffold. For most DGSS applications, either RE/TRE landscape data type (banding pattern or annotated information) would be sufficient for a high-resolution genetic surveillance/identification of specific genomes; however, a combinatorial interpretation of the two different RE/TRE landscape data types would be helpful for a final confirmation of the critical surveillance data sets (e.g., forensic identification for justice system). Interrogation of the RE/TRE landscape data, which are collected from individual genomes, via multi-dimensional (type, copy number, position, and time/space of REs/TREs) pattern-computation would allow for precise and dynamic (temporal and spatial) genetics surveillance and/or identification of all life forms.

Within each species, the genome-wide RE/TRE landscape information (type, copy number, position, and time/space) is expected to be variable depending on a host of factors (e.g., individual, organ, cell type, stress environment, and age), resulting in non-uniform genome sizes unique for the individual genomic DNA sources. For efficient storage, normalization, and computation of the multi-dimensional RE/TRE landscape information, which resides in these non-uniform and dynamic (temporal and spatial) genome platforms, we designed a novel genome-RE/TRE management system specifically designed for each species/individual, named the Universal Genome. The Universal Genome of each species/individual is able to encompass all genome variations (e.g., structure, sequence, and size) and it is expandable to accommodate newly obtained genetic information (e.g., REs, TREs, “TRE genes”, conventional genes) to their normalized relative positions. The Universal Genome permits efficient storage and annotation as well as rapid analyses of REs/TREs and other genetic elements (e.g., conventional genes, small RNAs) on the genomes of variable structures and sizes since the positions of each element are normalized into a single dynamically synchronized universal frame. For each species/individual, the DGSS will be built and operated on this dynamically adaptable and normalized Universal Genome scaffold.

The following highlight unique features of an exemplary DGSS:
Unbiased genetics surveillance/identification target (RE/TRE) types and loci: RE/TRE target information (type and locus) will be collected de novo as the amplicons are generated, and can be used for the unbiased surveillance/identification of specific genomes.
Combinatorial computation of key RE/TRE properties (type, copy number, position, and time/space of individual RE/TRE targets): Combinatorial computation of the multi-dimensional RE/TRE landscape information, which is collected de novo, enable high-resolution and precision surveillance/identification of specific genomes.
Highly tunable and/or customizable number of RE/TRE surveillance targets (type and locus): By employing different sets of RE/TRE surveillance targets, the genome surveillance/identification protocol can be customizable and surveillance results can be cross-checked.
Unmatched confidence in multi-dimensional RE/TRE landscape profile-based surveillance/identification of specific genomes due to the unbiased and high-resolution data characteristics.

Applications of DGSS

Applications of the DGSS technologies described herein, including the RE Arrays, include the following:

1. Introduction of the dynamic (temporal and spatial) and multi-dimensional RE/TRE landscape data (type, copy number, position, and optionally time/space of REs/TREs), which are directly linked to RE/TREs' inherent diversity and acquired activity, as critical elements for the development of a highly tunable precision genetics surveillance/identification for individual humans (including monozygotic twins), animals, plants, cells (e.g., cultured cells) and microbes;

2. Introduction of the dynamic (temporal and spatial) landscape data of repetitive element arrays (RE arrays), which are directly linked to REs/TREs' inherent diversity and acquired activity, as core elements for the development of precision genetics surveillance/identification for individual humans, animals, and plants;

3. Incorporation of both inherent and acquired RE/TRE landscape information into a life-long periodic or sporadic (incident-specific) genetics/health surveillance system using an individual's (e.g., human, animal, plant) personalized Universal Genome and

4. Identification and monitoring of cell types (e.g., cultured or primary cells) based on inherent and acquired RE/TRE landscape information on a species-specific Universal Genome scaffold.

5. Monitoring of genomic stability of cells grown in culture based on inherent and acquired TRE landscape information on a species-specific Universal Genome scaffold

6. Genomic monitoring/surveillance of cell differentiation processes (e.g., stem cells) based on inherent and acquired RE/TRE landscape information on a species-specific Universal Genome scaffold.

7. Monitoring and confirmation of crossing-over events between two different individuals/strains of humans, animals, or plants by examining crossing-over maps based on the inherent and acquired RE/TRE landscape information of parental strains and offspring on a species-specific Universal Genome scaffold. This would be especially important to monitor plant cross-breeding at early life stages.

8. Establishment of genetics surveillance system for laboratory animals of conventional-inbred and genetically engineered mammals or cells, e.g., mouse strains (e.g., CRISPR-CAS9-edits, transgenics, knock-outs) based on inherent and acquired RE/TRE landscape information of parental strains and offspring on a species-specific Universal Genome scaffold.

9. Establishment of a genetics surveillance system for genetically engineered/modified/edited plants (e.g., CRISPR-CAS9-edits, transgenics, knock-outs) based on the inherent and acquired RE/TRE landscape information of parental strains and offspring on a species-specific Universal Genome scaffold.

10. Monitoring and confirmation of stability and compatibility of the CRISPR-CAS9-edited cells (derived from humans, animals, and plants) by surveying the RE/TRE landscape profile on a species-specific Universal Genome scaffold.

11. Monitoring of the genomic stability of laboratory animals which are subjected to pharmacogenomics studies by examining changes in the RE/TRE landscape profile on a species-specific Universal Genome scaffold.

12. Identification and development of pathologic markers for studying various disease processes (e.g., cancer, aging-related disorders) by tracking the inherent diversity and acquired transposition activity of RE/TREs on a species- or individual (for temporal and spatial genomic changes within an individual)-specific Universal Genome scaffold.

13. Identification and development of diagnostic markers for diseases with unknown causative agents (e.g., cerebral palsy, autism spectrum disorder, allergy, susceptibility/resistance to specific diseases) or without any tangible diagnostic markers by tracking the inherent diversity and acquired transposition activity of RE/TREs on a species- or individual (for temporal and spatial genomic changes within an individual)-specific Universal Genome scaffold.

14. Development of non-conventional diagnostics systems by identifying genomic risk factors for a host of relatively well-characterized diseases (e.g., neonatal trisomy test), especially the ones without a reliable and/or efficient diagnostic tool available, such as celiac disease, Crohn's disease, and multiple sclerosis, by focusing on the inherent diversity and acquired activity of RE/TREs on a Universal Genome scaffold.

15. Identification and development of prognostic genomic signatures for a range of cancer types which predict differential cancer progression patterns, such as “DCIS (ductal carcinoma in situ)-forever” vs. “DCIS to breast tumor” based on the inherent and acquired RE/TRE landscape information on a species-specific Universal Genome scaffold.

16. Identification and development of prognostic genomic signatures for a range of aging-related disorders based on the inherent and acquired RE/TRE landscape information on a species-specific Universal Genome scaffold.

17. Temporal surveillance of the genome stability of a patient undergoing radiation therapy or chemotherapy by examination of changes in the RE/TRE landscape profiles and affected genomic regions within an individual-specific Universal Genome scaffold.

18. Surveillance of the effects of drugs and compounds on genome stability of a range of cultured cell types by examination of changes in the RE/TRE landscape profiles and affected genomic regions on a species-specific Universal Genome scaffold.

19. Surveillance of the effects of drugs and compounds on genome stability of experimental animals and human patients by examination of changes in the RE/TRE landscape profiles and affected genomic regions on a species-specific Universal Genome scaffold.

20. Temporal surveillance of the genome stability/variation of a patient who undergoes a series of acute disease episodes (e.g., trauma, infection) by examination of changes in the RE/TRE landscape profiles and affected genomic regions within an individual-specific Universal Genome scaffold.

21. Temporal surveillance of the genome stability and/or clonality of cancer patients (e.g. leukemia) undergoing treatment by examination of changes in RE/TRE landscape profiles within an individual-specific Universal Genome scaffold.

22. Development of the “RE/TRE landscape” biochip systems seeded with species/strain/cell type-specific multi-dimensional RE/TRE landscape information (type, copy number, position, and time/space of RE/TREs) annotated on a relevant Universal Genome scaffold for efficient surveillance of genome identity and/or stability.

23. Development of disease diagnostic systems based on the RE/TRE landscape biochip systems seeded with disease-specific multi-dimensional RE/TRE landscape information (type, copy number, position, and time/space of RE/TREs) annotated on the relevant species' Universal Genome scaffold.

24. Development of disease (e.g., inflammation) diagnostic systems based on the “TRE gene” biochip systems seeded with disease-specific RE/TRE gene sequences annotated on the relevant species' Universal Genome scaffold.

25. Establishment of an individual/strain/species-specific genome management and application systems (GMAS) to organize the constantly expandable DGSS and accompanying components (e.g., species-specific RE/TRE libraries) on the Universal Genome scaffold.

26. Establishment of disease-specific GMAS-DGSS which are enabled to learn and utilize newly annotated RE/TRE landscape and other relevant information.

27. Development and incorporation of genome modeling technologies within the GMAS-DGSS for efficient monitoring and determination of genomic phenotypes by multi-dimensional computation of RE/TRE landscape information (type, copy number, position, and space/time of RE/TREs).

28. The DGSS can also be used to determine the origin of a subject (e.g., where a migrating animal or a plant comes from). This is because RE/TREs' types, copy numbers, positions can be influenced by various environment factors as well. These factors include climate (e.g., temperature and amount of sunlight), food, and pollution, etc. Thus, in one aspect, the present disclosure provides methods of identifying the origin of a test subject. In some embodiments, the methods involve determining the type, position, and size of at least one repetitive element family in the genome of the test subject; comparing the type, position, and size of the repetitive elements of the test subject to the type, position, and size of the repetitive elements of a reference subject with known origin. If the type, position, and size of the repetitive elements are statistically different, it can be determined that the test subject and the reference subject have the different origins. If the type, position, and size of the repetitive elements are not statistically different, it can be determined that the test subject and the reference subject have the same origin.

29. The present disclosure also provides methods for clustering/sub-classifying a wide range of diseases (e.g., breast cancer, autism spectrum disorder). As used herein, the subject can be a human, an animal, or a plant. Various clustering algorithms can be used. Based on the clustering results, a person skilled in the art can identify a pattern that is unique to the individual clusters/subtypes of the disease. This pattern can be used to determine whether a test subject has the specific subtype disease.

30. The present disclosure also provides methods for cell line authentication with regard to identity, divergence, and contamination. Cultured cells are important for research (e.g., human cells, cancer cells). When it comes to interpreting results, knowing the origin of a cell line is imperative. However, cell lines can be mislabeled for various reasons, e.g., mix-up by accident. The present disclosure can be used to determine whether a test cell line belongs to a cell line of interest. In addition, multiple passages in a culture setting can lead to genome/DNA rearrangement. Thus, it is important to measure divergence of the cell lines' genomes in a culture setting. In some embodiments, the methods can also be used to determine whether the cell is from a male subject (e.g., a man, or a male animal) or from a female subject (e.g., a woman, or a female animal).

31. The methods described herein can also be used to determine whether a cell culture is contaminated by microorganisms (e.g., bacterium, virus, fungus). In these cases, the probes, which are designed to target the genome of microorganisms, can be mixed with the repetitive element probes employed for cell line authentication.

32. The methods described herein can be used in crossing over mapping in plants, animals, and humans. In these cases, the type and position information of the repetitive elements from gender-matching siblings/littermates will be compared against each other using their parents' genome as a reference. It is not necessary to have the parents' genome as a reference for the crossing over mapping. In some embodiments, the siblings are of the same sex (e.g., they are all brothers, or they are all sisters). In some embodiments, one sibling has the phenotype of interest (e.g., a disease), while the other does not. In some embodiments, the phenotype of interest is autism spectrum disorder, bipolar disorder, schizophrenia, or any diseases without known causative agents and/or genetic risk factors.

Other applications are also within the scope of the present disclosure.

### Repetitive Element Arrays (RE Arrays)

The present methods can include the generation and use of graphical representations of Repeat Element Arrays, which are species specific and ordered genome units. The arrays can be generated using unbiased self-alignment and dot-matrix plot visualization of the type (based on primary sequence), position (relative to the NCBI reference genome, for example), and size (e.g., length or number of repeats) of the REs or TREs. Each array may be representative of a specific time/space (e.g., a specific age of the cell or organism or a specific time in culture, or a specific tissue, organ, or organism source, or specific conditions under which the sample was originally obtained). For example, as shown in FIG. 19, to identify RE arrays, self-alignment of a query sequence was performed to mine direct (blue angle) REs and inverse (red angle) REs followed by dot-matrix presentation of their occurrences and positions. RE arrays are formed by combinatorial organization of occurrences and positions of direct and inverse RE sets.

The RE Arrays have a number of uses, as described herein; for example, the (known or unexplored) polymorphisms in species-unique RE arrays can serve as novel identifiers of genomes from a cell or organism, with extraordinary levels of resolution and precision. In addition, within a species, functional variations in RE array configurations could be directly applied to diagnostics as well as to the general studies of normal and disease biology. Furthermore, digital forms of RE arrays can be used as a “RE array code” to identify individual humans, animals, and plants.

The inherent diversity of RE arrays, and their responsiveness to acquired RE activity, makes them particularly useful for: 1) genome identification, 2) diagnostics, 3) studies of normal and disease biology, and 4) development of digital RE array ID.

The RE array can be stored, e.g., in electronic media such as a flash drive as well as on paper or other media. The RE array can also be represented electronically on a monitor or screen, such as on a computer monitor, a mobile telephone screen, or on a personal digital assistant (PDA) screen. The RE array can be further subjected visual or optical analysis and comparison, e.g., with a laser scanner or image capture device, such as a charge-coupled device (CCD). Images on paper or other non-electronic media can be scanned, e.g., digitally, and then compared by machine. For example, these images can then be compared using standard pattern recognition software, such as fingerprint matching or facial recognition programs. Alternatively, the RE Arrays can also be analyzed and compared by computer in digital, electrical form without the need for a tangible printout or image represented on a computer or other screen or monitor.

The RE arrays can be generated using a computer system, e.g., as described in WO 2011/146263 and FIG. 8 therein, which is a schematic diagram of one possible implementation of a computer system 1000 that can be used for the operations described in association with any of the computer-implemented methods described herein. The system 1000 includes a processor 1010, a memory 1020, a storage device 1030, and an input/output device 1040. Each of the components 1010, 1020, 1030, and 1040 are interconnected using a system bus 1050. The processor 1010 is capable of processing instructions for execution within the system 1000. In one implementation, the processor 1010 is a single-threaded processor. In another implementation, the processor 1010 is a multi-threaded processor. The processor 1010 is capable of processing instructions stored in the memory 1020 or on the storage device 1030 to display graphical information for a user interface on the input/output device 1040.

The memory 1020 stores information within the system 1000. In some implementations, the memory 1020 is a computer-readable medium. The memory 1020 can include volatile memory and/or non-volatile memory.

The storage device 1030 is capable of providing mass storage for the system 1000. In one implementation, the storage device 1030 is a computer-readable medium. In various different implementations, the storage device 1030 may be a disk device, e.g., a hard disk device or an optical disk device, or a tape device.

The input/output device 1040 provides input/output operations for the system 1000. In some implementations, the input/output device 1040 includes a keyboard and/or pointing device. In some implementations, the input/output device 1040 includes a display device for displaying graphical user interfaces.

The features described can be implemented in digital electronic circuitry, or in computer hardware, software, firmware, or in combinations of them. The features can be implemented in a computer program product tangibly embodied in an information carrier, e.g., in a machine-readable storage device, for execution by a programmable processor; and features can be performed by a programmable processor executing a program of instructions to perform functions of the described implementations by operating on input data and generating output. The described features can be implemented in one or more computer programs that are executable on a programmable system including at least one programmable processor coupled to receive data and instructions from, and to transmit data and instructions to, a data storage system, at least one input device, and at least one output device. A computer program includes a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. Computers include a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer.

The features can be implemented in a computer system that includes a back-end component, such as a data server, or that includes a middleware component, such as an application server or an Internet server, or that includes a front-end component, such as a client computer having a graphical user interface or an Internet browser, or any combination of them.

The components of the system can be connected by any form or medium of digital data communication such as a communication network. Examples of communication networks include, e.g., a LAN, a WAN, and computers and networks that form the Internet.

The computer system can include clients and servers. A client and server are generally remote from each other and typically interact through a network, such as the described one. The relationship of client and server arises by virtue of computer programs running on the respective computers and having a client-server relationship to each other.

The processor 1010 carries out instructions related to a computer program. The processor 1010 may include hardware such as logic gates, adders, multipliers and counters. The processor 1010 may further include a separate arithmetic logic unit (ALU) that performs arithmetic and logical operations.

### EXAMPLES

The invention is further described in the following examples, which do not limit the scope of the invention described in the claims.

### Example 1. Temporally and Spatially Acquired Genomic Landscapes of Endogenous Retroviruses in C57BL/6J Inbred Mice

In almost all biomedical research fields involving animals, it is critical to clearly define and confirm the genetic constancy of animals employed in a wide range of experimental models for studying gene function, toxicity of candidate compounds, diseases, and others (Austin et al., 2004; Maronpot, 2013). The definition and degree of the genetic constancy of animals, which are subjected to various experiments, are tunable depending on the specific aim(s) of individual studies. For example, when a candidate compound for therapeutic drug development is evaluated for its side effects and/or toxicities, the genetic constancy of the experimental animals does not have to be highly stringent. Conversely, the majority of studies, which focus on understanding functions of genes using a pair of defective/mutated (conventional or targeted) and its matching control animals, rely on stringent genetic constancy for a proper evaluation of the experimental outcomes.

It is not uncommon to encounter variations in morphologic phenotypes among a population of a specific inbred mouse strain, such as C57BL/6J (Niu and Liang, 2009). Some of these phenotypic variations within individual inbred mouse populations are explained primarily by irreversible genetic drift events due to the genetic fixation of accumulated mutations, which are often discovered serendipitously or as outcomes of troubleshooting experiments (Taft et al., 2006). Taft et al. stated that the current repertoire of gene SNPs and other DNA markers (e.g., microsatellite elements) is not sufficient for screening genetic drift in mice (Taft et al., 2006). To circumvent the detrimental effects of cumulative genetic drifts over-time, key research mouse producers implemented control programs, such as the Genetic Stability Program (Jackson Laboratory) and the Genetic Monitoring Program (Taconic Biosciences, Inc). One of the key shared features of these programs is the cryopreservation of embryos as a future replacement of foundation mice. Jackson Laboratory reported that “Inbred strains within this program effectively remain genetically unchanged for at least the period of the program (projected 25 years)” (Taft et al., 2006).

As described herein, the reference mouse genome sequence (derived from C57BL/6J inbred mice) housed at the NCBI database has not yet been completely sequenced (Consortium, 2002). In addition, the NCBI's reference mouse chromosome Y, which was estimated to be ˜16 million nucleotides in length up until early 2013, is now annotated to have ˜92 million nucleotides. These findings summarize the difficulties which are inherent to understanding genome/chromosome biology as well as decoding/sequencing the entire genetic information system of humans and animals. In addition to the estimated 20,000 conventional genes annotated in the reference mouse genome, the vast majority of the mouse and human genomes are occupied by a plethora of TREome members. In contrast to conventional genes, the TREome is inherently and highly diverse within the mouse as well as human populations (Batzer et al., 1996; Bennett et al., 2004; Boissinot et al., 2004), unpublished). In addition, it has been well-documented that some members of the TREome have “gene” sequences which code for functional proteins, such as the superantigen of mouse mammary tumor virus type-ERVs and human endogenous retroviruses (Holder et al., 2012; Lee et al., 2014; Schmitt et al., 2015). Furthermore, certain TREome members of mice and humans respond to a range of stressors, leading to an increase in their activity (Antony et al., 2011; Cho et al., 2008b). Acquired TREome (e.g., MLV-ERVs) activities during the life course of an inbred mouse could involve five critical processes: 1) DNA-dependent RNA polymerization (transcription), 2) protein synthesis from TREome genes, 3) RNA-dependent DNA polymerization (reverse transcription) in the cytoplasm, 4) virion assembly, and 5) “random” integration of a DNA copy into the genome. One of the critical impacts imposed by the accumulation of acquired TREome activities would be alterations in the TREome landscapes in the affected genomes.

In this study, we examined the extent of variation in the configurations among the inherent germ-line and acquired (temporally and spatially) somatic genomes of C57BL/6J inbred mice using murine leukemia virus-type ERV (MLV-ERV) sequences as a TREome landscaping probe. The findings from this study provide evidence that: 1) with regard to the TREome landscapes, inherent diversity is visible among the population of C57BL/6J inbred mice evidenced by the variations in the TREome landscapes of germ-line DNAs, 2) there are spatial variations in the TREome landscapes among different organs/tissues within the individual mice, probably due to the dynamic accumulation of acquired activity of certain TREome members, 3) in particular, there are more copies of MLV-ERV in the kidney genomes of 19-month old C57BL/6J male mice compared to the liver genomes of the same mice, 4) there are gender specific TREome landscapes, suggesting the TREome's association with gender-specific phenotypes, and 5) distinct patterns of variations in the TREome landscape exist among the population of C57BL/6J inbred mice. One can assume that the entire, or at least the majority, of the information embedded in the genome of humans and mice participate in determining phenotypic details. In that case, surveillance of the inherent diversity and acquired activity of the TREome in the dynamic genomes of C57BL/6J inbred mice, as demonstrated in this study, would provide critical and valuable information for understanding the relationships between the genetic characteristics and phenotypes of inbred mice or research animals in general. We suggest that a mouse genetics surveillance system be established for a range of laboratory mouse strains which focuses on the inherent diversity and acquired activity of the TREome in conjunction with temporal and spatial variations in TREome landscapes. This somewhat unbiased TREome-based genetics surveillance system would serve as a synergistic tool for the current monitoring systems (e.g., Genetic Stability Program, Genetic Monitoring Program) which primarily rely on the cryopreservation of embryos and survey for polymorphisms of conventional genes and microsatellites in the absence of complete reference mouse genome sequences. Successful development and implementation of the TREome-based mouse genetic surveillance system would be applied for high-resolution genetic identification and monitoring of wide-ranging species, such as plants and their products, animals and their products, and humans which all harbor their own TREomes.

Materials and Methods

Animal Experiments

C57BL/6J inbred mice (females and males) of varying ages were purchased from the Jackson Laboratory (Bar Harbor, Me.; West Sacramento, Calif.) or obtained from Dr.

David Pleasure at the University of California, Davis (UC Davis). All animals were provided with water and food ad libitum during their housing at an UC Davis facility where some of them were aged for an extended period of time. The animal experiment protocol was approved by the Animal Use and Care Administrative Advisory Committee of UC Davis. Animals were sacrificed by CO₂ inhalation to collect sperm and/or tissues followed by snap-freezing in liquid nitrogen.

Genomic DNA Isolation and TREome (MLV-ERV) Landscaping by Inverse-PCR (I-PCR) Analyses

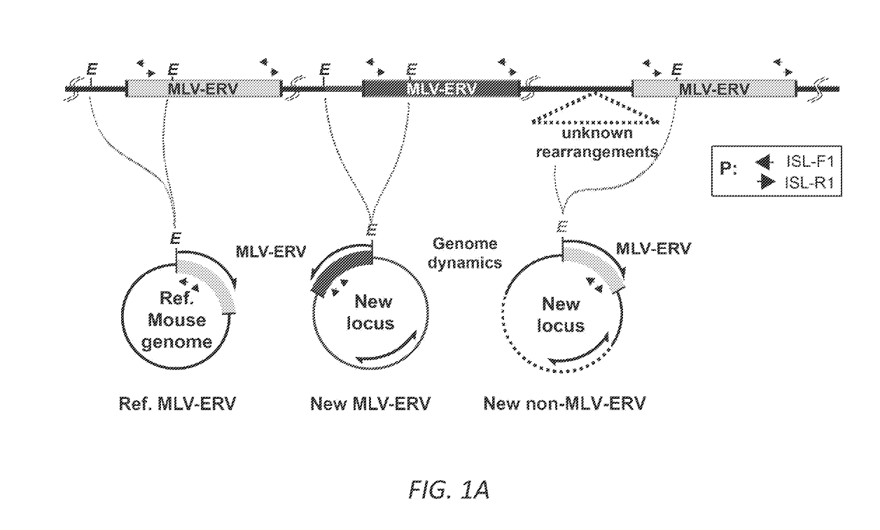

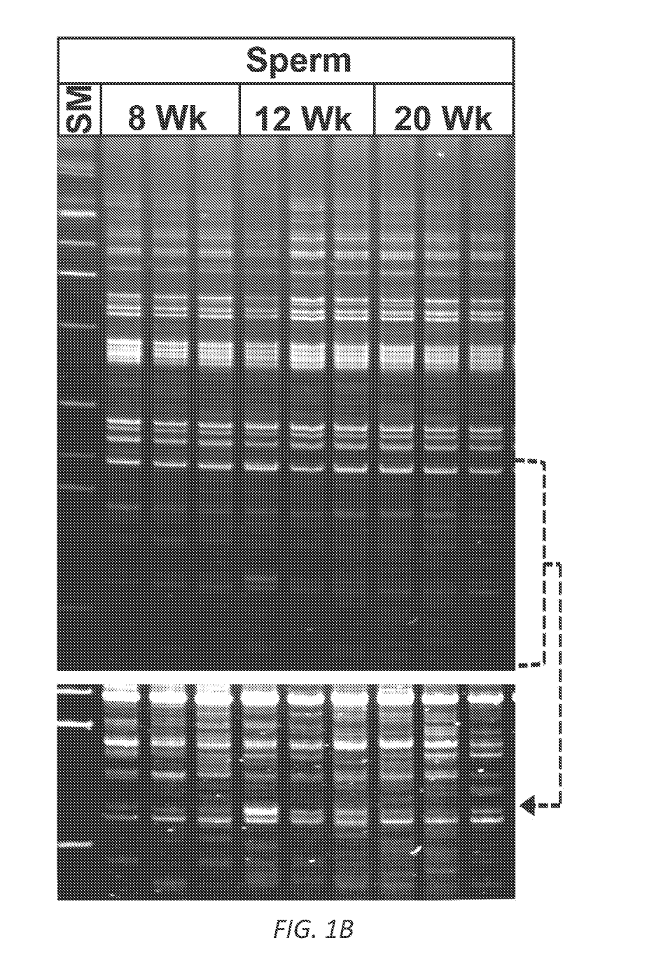

Snap-frozen sperm and somatic tissue samples were subjected to genomic DNA isolation using a DNeasy Tissue kit (Qiagen, Valencia, Calif.) and DNA samples were normalized to 20 ng/μ1. As an initial step for the I-PCR analyses (FIG. 1A), genomic DNAs (300 ng) were digested with Nco-I (New England Biolab, Ipswich, Mass.) at 37° C. for 4 hours followed by self-ligation of the digests using T4 ligase (Promega, Madison, Wis.) overnight at 4° C. The TREome landscape information was collected by I-PCR amplification of the junctions spanning putative MLV-ERV integration loci using 2 μl of the ligation products, Taq polymerase (Qiagen), and a pair of inverse primers designed from the conserved MLV-ERV sequences. The primer sequences and PCR condition are listed in Table 1. I-PCR products were resolved on a 7.5% polyacrylamide gel followed by ethidium bromide staining for visualization.

TABLE 1
PCR primers and reaction conditions.

| | | | PCR conditions | | | |
|---|---|---|---|---|---|---|
| Target | Primer | Sequence | Denaturation | Annealing | Elongation | Cycles |
| MLV-ERV | ERV-U1 | 5′-CGGGCGACTC | 95° C./ 30 | 55° C./ 60 | 72° C./ 60 sec | 30 |
| LTR | | AGTCTATCGG-3′ | sec | sec | | |
| | | (SEQ ID NO: 1) | | | | |
| | ERV-U2 | 5′-CAGTATCACCA | | | | |
| | | ACTCAAATC-3′ | | | | |
| | | (SEQ ID NO: 2) | | | | |
| Mouse Y | Mus-SRY | 5′-TGGGACTGGTG | 95° C./ 30 | 55° C./ 60 | 72° C./ 60 sec | 33 |
| Chromosome | 1B | ACAATTGTC-3′ | sec | sec | | |
| | | (SEQ ID NO: 3) | | | | |
| | Mus-SRY | 5′-GAGTACAGGTG | | | | |
| | 2B | TGCATGGAG-3′ | | | | |
| | | (SEQ ID NO: 4) | | | | |
| MLV-ERV | ISL-F1 | 5′-GACTGAGTCGC | 95° C./ 30 | 58° C./ 60 | 72° C./ 120 | 32 |
| LTR | | CCGGGTA-3′ | sec | sec | sec | |
| (I-PCR) | ISL-R1 | (SEQ ID NO: 5) | | | | |
| | | 5′-GCGGTTGAGAA | | | | |
| | | TACAGGGTC-3′ | | | | |
| | | (SEQ ID NO: 6) | | | | |
| MMTV-ERV | MTV-1B | 5′-TGCCGCGCCTG | 95° C./ 30 | 55° C./ 60 | 72° C./ 60 sec | 33 |
| SAg | | CAGCAGAAATG-3′ | sec | sec | | |
| | | (SEQ ID NO: 7) | | | | |
| | MTV-2A | 5′-TGTTAGGACTGT | | | | |
| | | TGCAAGTTTACTC- | | | | |
| | | 3′ (SEQ ID NO: 8) | | | | |

Real-Time Genomic DNA PCR Analyses of MLV-ERV Copy Numbers

For the kidney and liver genomic DNAs isolated from six 19-month old mice (3 females and 3 males), real-time genomic DNA PCR was performed in triplicate using a MX3005P instrument (Stratagene, Santa Clara, Calif.) with a reagent kit (Brilliant SYBR Green QPCR Master Mix) from Agilent (Santa Clara, Calif.) and 25 ng of each genomic DNA in triplicate. Details for the primers and PCR conditions are listed in Table 1.

Copy Number Calculation and Statistical Analysis

The results from quantitative real-time DNA PCR analyses of MLV-ERVs were calculated as a relative copy number per single copy of the hypoxanthine phosphoribosyl transferase (HPRT) gene using a modified delta-delta CT method (2̂(CT_{(HPRT)}−CT_{(MLV-ERV)})) (Livak and Schmittgen, 2001). A one-way ANOVA was used to determine the significance of differences in relative MLV-ERV copy number values between individual pairs of groups. Statistical significance was indicated when the P value is less than 0.05.

Results

Germ-Line Variations in the Genome-Wide TREome Landscapes Among C57BL/6J Inbred Mice

Current understanding of inbred mouse genetics projects that the genomic configuration of germ-line cells from mice of an inbred strain are virtually identical (Beck et al., 2000). Snapshots of the TREome landscapes of the germ-line (sperm) genomic DNA samples isolated from three age groups (8-, 12-, and 20-weeks) of C57BL/6J inbred mice were taken using a conserved region of MLV-ERVs as a probe (FIG. 1B). Although these TREome landscaping protocols, which employ restriction digestion and I-PCR, were designed for unbiased amplification of regions spanning the MLV-ERVs' insertion loci, it is possible that the I-PCR products include other genomic areas. The germ-line TREome landscapes of all nine mice (3/age group) shared a visible pattern of I-PCR amplicons; however, a close examination revealed that the TREome landscape pattern is unique for each mouse without any age group-specific patterns (FIG. 1B). For instance, the first mouse of the eight week-olds and the third mouse of the 20 week-olds had distinct I-PCR amplicon patterns compared to the others within the individual age groups. The results obtained from this experiment suggest that configurations of germ-line genomes are variable among C57BL/6J inbred mice with regard to the genome-wide TREome landscapes.

Spatial Variations in the TREome Landscapes Among Germ-Line and Somatic Genomes in a Single C57BL/6J Inbred Mouse

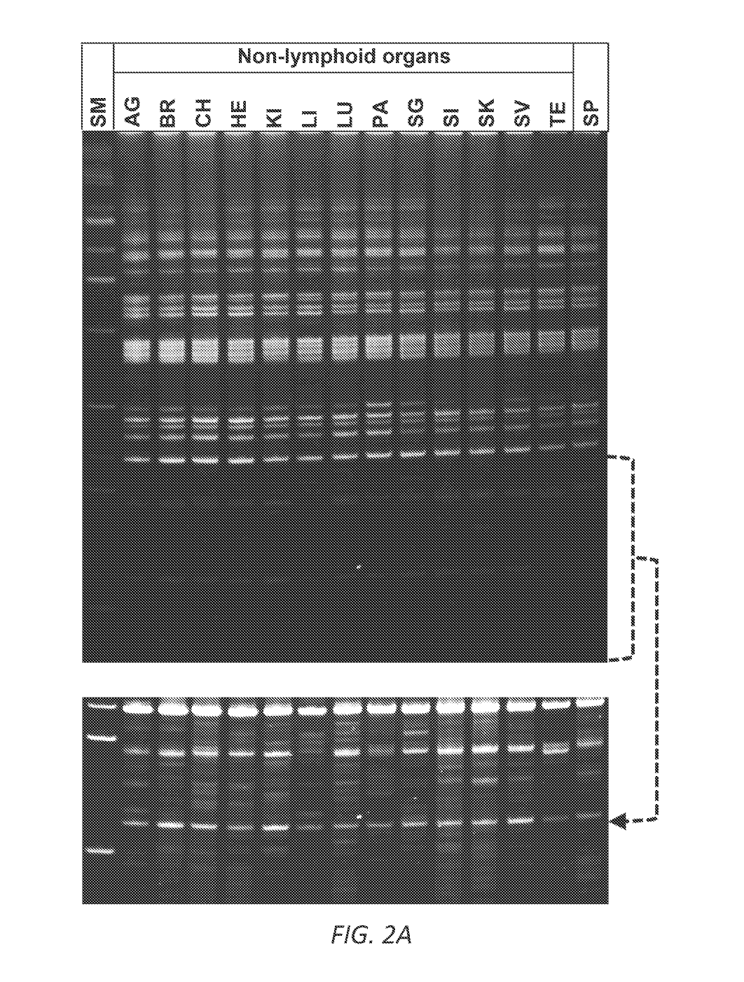

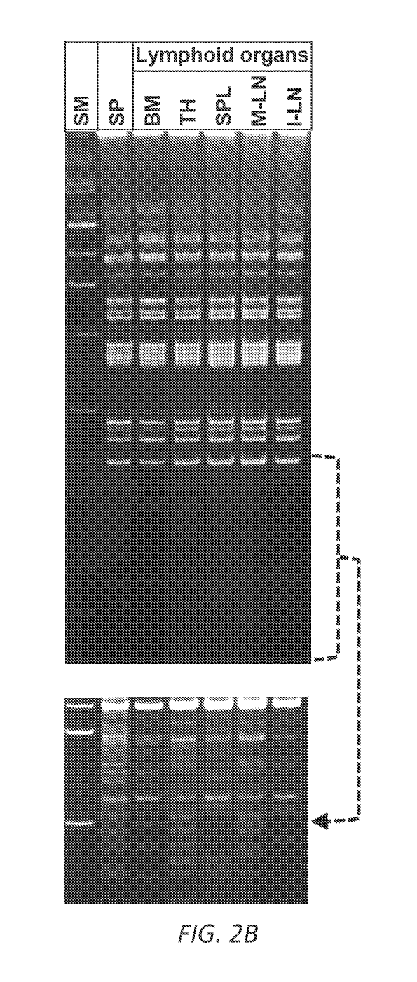

It is widely accepted that there are no significant changes in genome configuration, primarily in regard to the number and position information of nucleotides, during development and/or differentiation of an individual mouse or human (Giachino et al., 2013; Walsh et al., 1998). In this experiment, we investigated whether the structural configuration of the germ-line genome of an individual diversifies during development and/or differentiation, resulting in a pool of disparate TREome landscapes within somatic genomes. Genome-wide TREome landscapes of a set of 18 different somatic organs (13 non-lymphoid and 5 lymphoid) and sperm collected from a single male C57BL/6J inbred mouse (one of the 12-week olds above) were analyzed to examine spatial genomic variations within an individual. The TREome landscapes of the 13 non-lymphoid organs were highly variable and they were also different from the pattern of sperm although the profile of about a dozen I-PCR amplicons was shared among all of them (FIG. 2A). In addition, an examination of a group of five lymphoid organs (bone marrow, thymus, spleen, mesenteric lymph node, and inguinal lymph node) demonstrated marked variations in patterns of I-PCR amplicons (FIG. 2B). It is expected that genomic configurations of lymphoid organs (both primary and secondary) are different from germ-line cells due to extensive rearrangements during lymphoid cell development. Overall, some organs had more I-PCR amplicons than the others, and no unique banding pattern of I-PCR amplicons, which can differentiate the genomes of somatic organs from germ-line genomes, were identified. These findings indicate that genome-wide TREome landscapes of a single mouse are spatially diversified from the germ-line configurations depending on organ type (and potentially cell type), creating a pool of somatic variants.

Polymorphic TREome Landscapes in Different Brain Compartments of C57BL/6J Inbred Mice

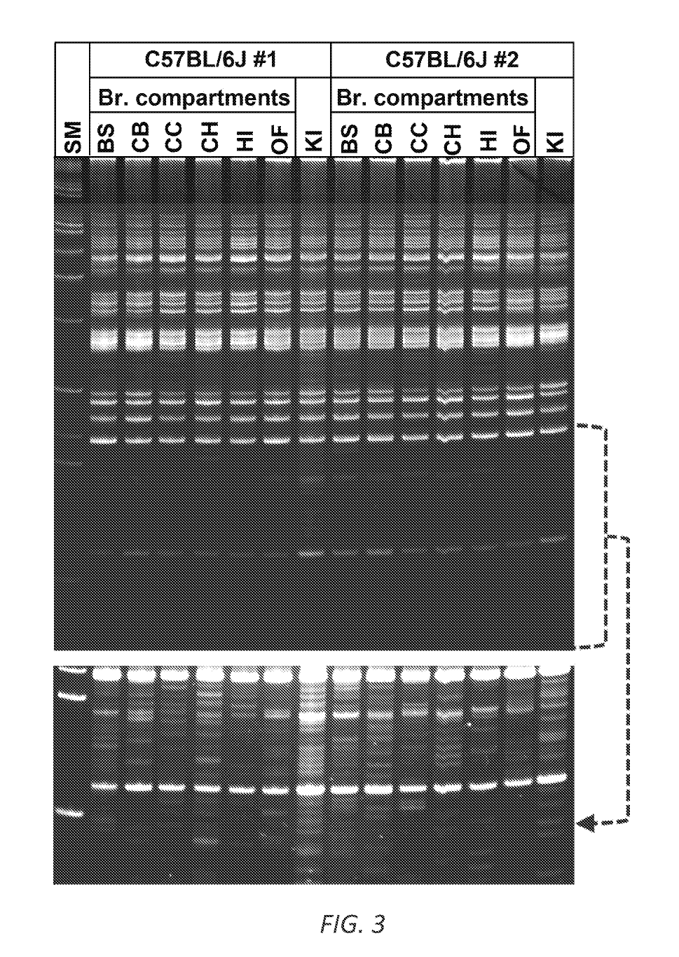

To examine whether there are spatial variations in TREome landscapes in the brain, genomic DNA isolated from six different brain compartments (brain stem, cerebral cortex, corpus callosum, cerebellar hemisphere, hippocampus, and olfactory bulb) of C57BL/6J female mice (5-week old) were subjected to the I-PCR landscaping analysis (FIG. 3). There were noticeable variations in the I-PCR amplicon patterns among the six brain compartments, and within each compartment, the TREome landscape patterns were not shared by the two mice examined. It is likely that the I-PCR amplicon patterns obtained from this experiment are not specific for the individual brain compartments, but each pattern rather represents temporal and spatial variations in the TREome landscapes in the genomes of a plethora of different cell types and/or cells in the brains of inbred mice.

Temporal Variations in TREome Landscapes in the Primary and Secondary Immune Organs of C57BL/6J Inbred Mice

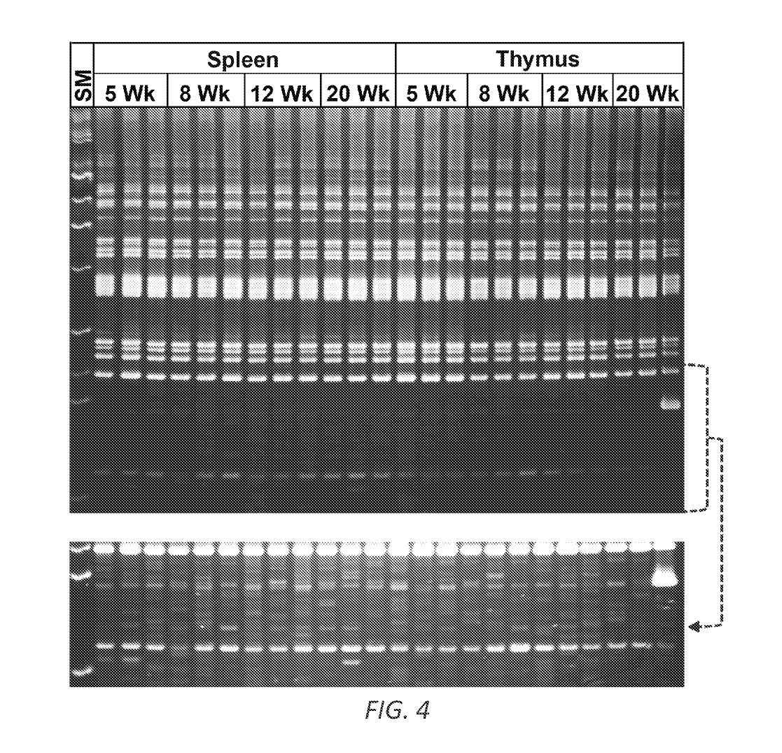

The cells in immune organs, such as thymus (primary) and spleen (secondary), are constantly subjected to a wide range of intrinsic and external stressors, some of which may have the potential for stimulating TREome activity. Using the I-PCR protocol, we examined whether TREome landscapes of the thymus and spleen are temporally altered with specific patterns in four age groups (5-, 8-, 12-, and 20-weeks) of C57BL/6J male mice. Substantial variations were observed in the TREome landscapes in both types of immune organs among all four age groups of mice; however, no age group-specific or immune organ-specific I-PCR amplicon patterns were discernible (FIG. 4).

Interestingly, the thymic TREome landscapes of the third mouse of the 20-week olds contained one unusually strong I-PCR amplicon band (FIG. 4) for which a follow-up investigation would be justified. It is likely that an additional set of landscaping probes may be needed to collect high-resolution data sets which allow for potential identification of age group- and/or immune organ-specific TREome landscapes.

Variations in TREome Landscapes of Spatially Separated Organ Sets in C57BL/6J Inbred Mice

Certain types of organs (e.g., lymph nodes, mammary glands) in humans and mice are found in more than one location (e.g., left side, right side). In this experiment, we examined variations in TREome landscapes in a set of three lymph nodes (thoracic mammary, inguinal mammary, and mesenteric), a pair of mammary fat pads (#2-right and #4-right), and a pair of bone marrow samples (derived from left and right femurs) isolated from a 27-month old female mouse. Examination of I-PCR amplicon profiles revealed substantial variations in TREome landscapes among the individual sets of three lymph nodes and two mammary fat pads (FIG. 5). In comparison to the lymph node and mammary fat pad sets, the two bone marrow samples isolated from left and right femurs had highly similar I-PCR amplicon patterns. Furthermore, it was interesting to observe that TREome landscapes of the two bone marrow samples have unique I-PCR amplicon profiles with significantly lower densities compared to the lymph nodes and mammary fat pads. The fact that the population of cells in the bone marrow (primary lymphoid organ) is considered to be less differentiated compared to the cells in the lymph nodes (secondary lymphoid organ) may attribute to the differences in density of the TREome landscapes. In fact, the cells in the lymph nodes, which are constantly subjected to a host of stressors, are expected to maintain elevated levels of TREome activity, leading to an increase in the number of TREome positions in the affected genomes. The findings from this study demonstrate spatial variations in TREome landscapes among same organ types at different locations.

Gender- and Individual-Specific Variations in TREome Landscapes in C57BL/6J Inbred Mice

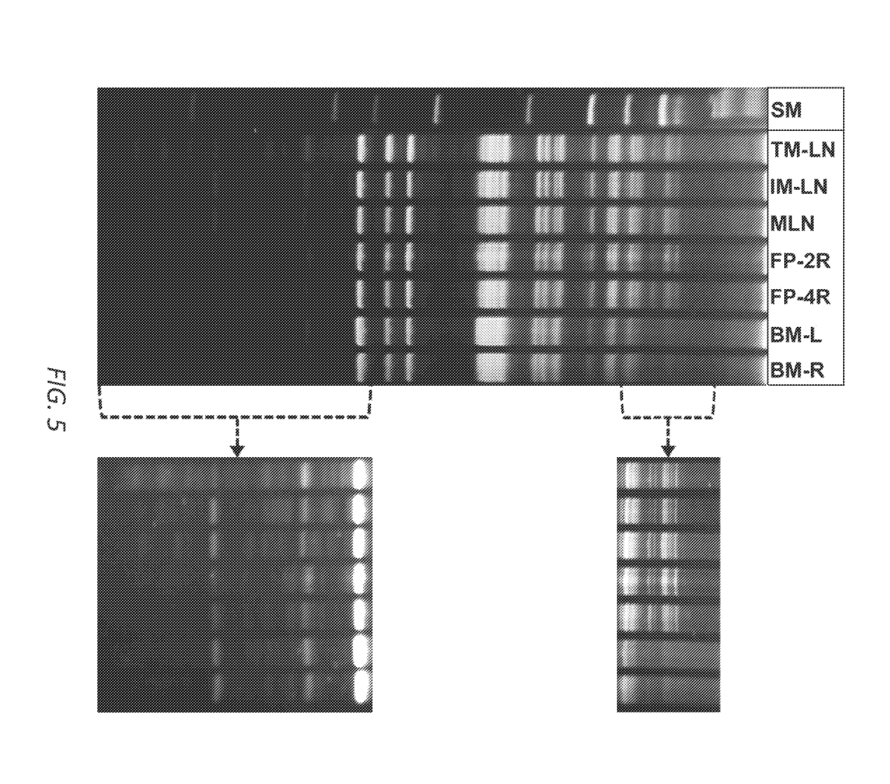

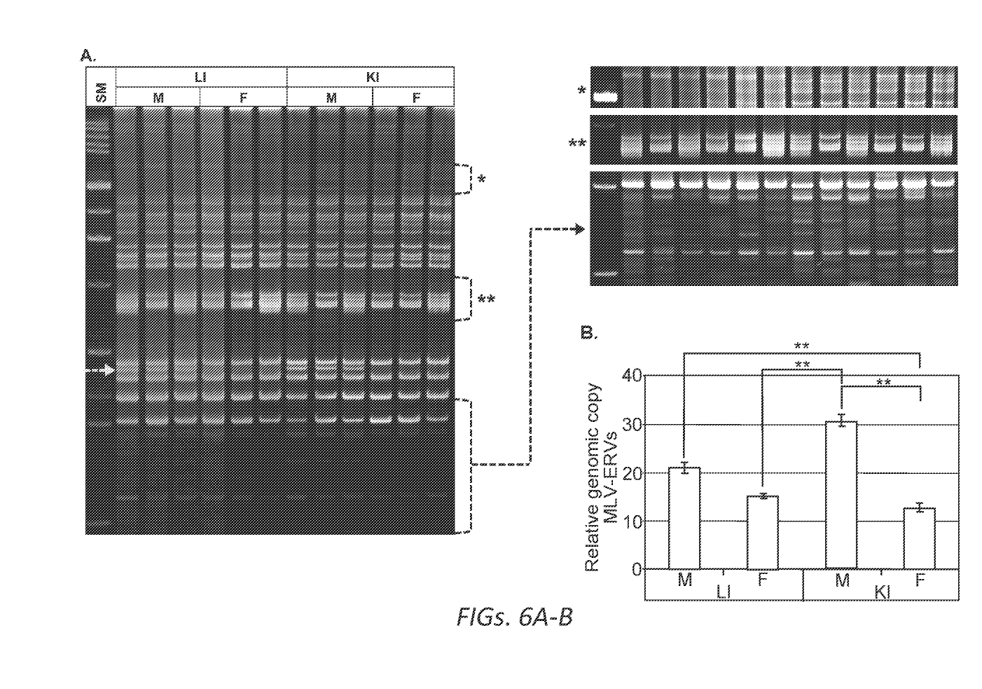

It has been reported that chromosome Y of C57BL/6J male mice is densely populated with a plethora of repetitive elements (Lee et al., 2013). In this experiment, we examined the differences in TREome landscapes between three females and three males using the kidney and liver samples from 19-month old C57BL/6J mice. Among other variations, there was one distinct I-PCR amplicon band which is found only in male mice in both tissues (FIG. 6A); thus, presumably, the male-specific amplicon band is derived from the chromosome Y. A close examination of the banding patterns revealed that TREome landscapes of the liver genomes from all six mice (three females and three males) lack a cluster of amplicon bands which are clearly present in all six kidney genomes (FIG. 6A-*). Unexpectedly, two distinct clusters of I-PCR amplicon bands were identified in the TREome landscapes of both kidney and liver genomes. Interestingly, each of the six mice had either one, but not both, of the two cluster patterns in its TREome landscape, regardless of its gender identity (FIG. 6A-**). This data provides another set of solid evidence that genomic configuration with regard to the TREome landscape is visibly variable depending on the individual C57BL/6J inbred mice within the same gender. Furthermore, to quantify differences in the copy number of the TREome (MLV-ERVs) between females and males, real-time PCR analysis was performed using genomic DNAs of the kidneys and livers. As somewhat expected, in both kidney and liver genomes, male mice had higher copy numbers of MLV-ERVs compared to female mice (FIG. 6B). Interestingly, within the three male mice, the kidney genomes had substantially more MLV-ERV copies compared to the liver genomes whereas there were no significant differences in female mice. In consideration of the relatively old age (19-months) of the mice examined in this study, it would be interesting to repeat the same experiment with a series of age groups from neonates to two-year olds. A future investigation which focuses on mapping putative additional MLV-ERV loci in the kidney genomes, in comparison to the liver genomes, of these male mice may provide a novel insight into kidney biology.

### Example 2. Genomic Landscapes of Endogenous Retroviruses Unveil Intricate Genetics of Conventional and Genetically-Engineered Laboratory Mouse Strains

Currently, the normal and disease biology of laboratory mice and humans is explained primarily in the context of the function and polymorphism of conventional genes. Thus far, the majority of conventional gene-based attempts to decode the tangible mechanisms of normal and disease states and to identify diagnostic markers have been inconclusive or unsuccessful (Padyukov, 2013; Seok et al., 2013; Takao and Miyakawa, 2015a; Takao and Miyakawa, 2015b). Reportedly, laboratory mice and humans share ˜80% of conventional gene sequences (Consortium, 2002; Guenet, 2005); this is inconsistent with the notion that phenotypic details are primarily determined by conventional genes when dramatic phenotypic distances exist between the two species. Considering the limited understanding of the complete genome information system of mice and humans, conventional gene-focused approaches would be insufficient for decoding the enormous scope of phenotypic details and their variations. The inherent TREome diversity of individual laboratory mouse strains is directly associated with the polymorphic protein coding potentials for TREome genes. Whereas the acquired activity of the inherently diverse TREome may play a role in the fine-tuning the function of TREome genes as well as conventional genes, which reside near the new TREome positions, through their networks of transcription regulatory elements, contributing to strain-specific phenotypic details (Amid et al., 2009; Giardine et al., 2007).

The intricate and unexplored variations in the genomes of laboratory mouse strains are directly associated with two distinct, but interrelated, characteristics of the TREome. First, the information embedded in the TREome landscape of a mouse strain, which is defined by TRE information regarding type, copy number, and position, can be examined to understand how conventional gene(s) neighboring a genomic position of a specific TRE type is regulated. The finding from this study that TREome landscape patterns are different between the C3H/HeJ strain and its TLR4 wildtype control strain (C3H/HeOuJ) needs to be investigated further to determine whether the differences are linked to the expression of certain gene(s) other than TLR4 (Kamath et al., 2003). Similarly, confirmation of the impact of differences in the TREome landscapes between the CD14 knock-out and its backcross-control strain (C57BL/6J) on the expression of genes outside of the knock-out locus is deemed to be necessary. It likely that some other knock-out and/or transgenic mouse strains need to be subjected to similar scrutiny of their genomic configurations with regard to the TREome landscape, in comparison to their control strains. Second, there are numerous and highly polymorphic TREome genes in the genomes of laboratory mouse strains which have not been fully identified, accounted for, or understood. Tangible coding potentials of TREome genes could be either presumed full-length or variable in length due to introduction of mutations over time. It has been demonstrated that certain TREome genes, such as the envelope genes of MLV-ERVs and MMTV-ERV SAg genes, play functional roles in biological processes (Bentvelzen, 1992; Huber et al., 1994; Kotzin et al., 1993). In addition, TREome (human endogenous retroviruses) gene isoforms isolated from a human burn patient's genomic DNA demonstrated differential potentials for regulating inflammatory mediators, such as IL-6 and IL-1β (Lee et al., 2014). Despite the previous studies which reported a range of functionality of TREome genes in both mice and humans, unfortunately, they are often called non-coding long RNAs in current literature (Geisler and Coller, 2013; Gibb et al., 2015). In this study, polymorphisms in TREome genes in laboratory mouse strains are reflected in the identification of 183 isoforms of the MMTV-ERV SAg gene which was reported to play a critical role in shaping the systemic immune cell profile (Acha-Orbea and MacDonald, 1995; Kotzin et al., 1993; Tomonari et al., 1993). The differences in the profile of MMTV-ERV SAg gene isoforms between the C3H/HeJ strain and its TLR4 control (C3H/HeOuJ) suggest that a specific immune system would be developed within each strain due to the activity of a unique set of MMTV-ERV SAg genes, especially during T lymphocyte selection events. In order to confirm the data obtained from the TLR4 studies using C3H/HeJ and its control (C3H/HeOuJ) strains, the potential impacts of the differential MMTV-ERV SAg activities on immune function should to be examined.

Despite the absence of a single reference mouse genome, which is completely sequenced, it is often stated that the population of laboratory mouse strains share a high level of genome sequences (Frazer et al., 2007; Kirby et al., 2010; Mekada et al., 2009). In addition, a recent change in the putative size of the NCBI's reference mouse chromosome Y from ˜16 Mb (Build 37.2) to ˜92 Mb (Annotation Release 105) suggests that more time is needed to confirm the size of each chromosome within individual laboratory mouse strains (Lee et al., 2013). In spite of the lack of the full sequence information from a single reference strain, current genetic monitoring systems for laboratory mice rely primarily on polymorphism data derived from limited sets of conventional genes and microsatellites to determine genetic uniformity/status/identity of a specific strain/substrain. The finding that the TREome (MLV-ERVs) sequences are more variable among 12 laboratory mouse strains, in comparison to conventional gene sequences, suggests that the TREome landscape contributes to the formation of unique phenotypic characteristics embedded in each strain. Furthermore, the discrepancy in TREome landscapes between the CD14 knock-out and its backcross-control strain (C57BL/6J) informs that all genetically engineered mouse strains may need to be examined to confirm the genetic uniformity with their matching controls, outside of the individual targeted loci. On the other hand, the unexplained/unexpected phenotypic variations, which are frequently encountered in genetically engineered mouse strains, such as runt or normal weight of STAT-1−/− mice (Bona and Revillard, 2001; Kim et al., 2003), could be explained by checking the genomic configuration of the engineered mouse population. In certain circumstances, confirmation of uniformity within the entire genomes (minus targeted loci) may be necessary to validate the results collected from studies involving genetically engineered mouse strains.

Materials and Methods

Animal Experiments

The following mouse strains were purchased from the Jackson Laboratory: female C57BL/6J, C3H/HeJ, C3H/HeOuJ, and CD14 knock-out (B6.129S4-Cd14^{tm/frm}/J). All animals were provided with water and food ad libitum at an UC Davis facility and some of the mice were aged for a period of time. The experimental protocol was approved by the Animal Use and Care Administrative Advisory Committee of UC Davis. Animals were sacrificed to collect tissues followed by snap-freezing in liquid nitrogen.

Genomic DNA of Various Laboratory Mouse Strains

Snap-frozen tissue samples were subjected to genomic DNA isolation using a DNeasy Tissue kit (Qiagen, Valencia, Calif.) and the DNA samples were normalized to 20 ng/μl. In addition, genomic DNA from 63 laboratory mouse strains, which include nine 129 sub strains, were purchased from the Jackson Laboratory (Bar Harbor, Me.). In addition, genomic DNA from a C57BL/6J×129S1/SvlmJF2/J (B6129SF2/J) mouse, a F2 hybrid from F1×F1 whose parents were C57BL/6J (female) and 129S1/SvlmJ (male), was obtained from the Jackson Laboratory. According to the information from the Jackson Laboratory's web site, the genomic DNA was isolated from either the brain or spleen of respective mouse strains. Gender identity of each DNA sample was confirmed by amplifying a region specific for mouse chromosome Y by PCR using a pair of primers (Table 2) followed by agarose gel electrophoresis.

TABLE 2
PCR primers and reaction conditions.

| | | PCR condition | | | |
|---|---|---|---|---|---|
| Primer | Sequence | Denaturation | Annealing | Elongation | Cycles |
| ISL-F1 | 5′-GACTGAGT | 94° C. / | 58° C. / 60sec | 72° C. / | 32 |
| | CGCCCGGGTA-3′ | 30sec | | 120sec | |
| | (SEQ ID NO: 5) | | | | |
| ISL-R1 | 5′-GCGGTTGAG | | | | |
| | AATACAGGGTC-3′ | | | | |
| | (SEQ ID NO: 6) | | | | |
| ERV-U1 | 5′-CGGGCGACTC | 95° C. / | 55° C. / 60sec | 72° C. / | 40 |
| | AGTCTATCGG-3′ | 30sec | | 60sec | |
| | (SEQ ID NO: 1) | | | | |
| ERV-U2 | 5′-CAGTATCACCA | | | | |
| | ACTCAAATC-3′ | | | | |
| | (SEQ ID NO: 2) | | | | |

Polymorphism Analysis of Genomic TREome (Murine Leukemia Virus-Type Endogenous Retrovirus [MLV-ERV]) Long Terminal Repeats (LTRs)

The polymorphic regions of the MLV-ERV LTRs were identified from the genomic DNAs of 12 laboratory mouse strains (Jackson Laboratory) by PCR using a set of primer pairs (Table 2) which were designed from a well-conserved region. Following ligation into a TA vector (Promega, Madison, Wis.), 24 colonies were picked from the MLV-ERV amplicons of each strain and plasmid DNAs were prepared using a QIAprep spin Miniprep kit (Qiagen) before sequencing (Molecular Cloning Laboratories, South San Francisco, Calif.). A set of unique MLV-ERV LTR sequences was compiled for each mouse strain by multiple alignment analysis using the Vector NTI program (Invitrogen, Carlsbad, Calif.). Within a set of unique MLV-ERV LTR sequences for each mouse strain, the occurrence frequency of 64 four-nucleotide “word” (a nucleotide sequence of specific length) combinations at all four possible reading frames were counted using a program we developed. Within each strain, the occurrence frequency data for the individual words were normalized and converted into probability distribution function (PDF) values. For each word, the average and standard deviation of the PDF values from all 12 strains were calculated using Excel (Microsoft, Redmond, Wash.). Based on an assumption that the higher the standard deviation in a word, the more variation in the word, the extent of variations in each four-nucleotide word within the 12 strain-population was visualized with a schedule of gray shades (white-lowest variation; black-highest variation) on a 16×16 (=256) matrix. To examine/simulate diversity in conventional gene sequences in comparison to the MLV-ERV LTR sequences, the single nucleotide polymorphism (SNP) data for the GAPDH gene (˜4.7 Kb) among 19 laboratory mouse strains (A/J, C57BL/6J, 129X1/SvJ, AKR/J, BALB/cByJ, C3H/HeJ, CAST/EiJ, DBA/2J, FVB/NJ, MOLF/EiJ, NOD/ShiLtJ, SM/J, BTBR T<+>Itpr3<tf/J, KK/H1J, LG/J, NZW/LacJ, PWD/PhJ, WSB/EiJ, and 129S1/SvImJ) was subjected to the same PDF analysis as above.

TREome Landscaping of Mouse Genomes Using MLV-ERV Sequences as a Probe

Genomic DNA (20 ng) was cut with Nco-I (New England Biolab, Ipswich, Mass.) at 37° C. for 4 hours followed by self-ligation of the cut fragments using T4 ligase (Promega) overnight at 4° C. The TREome landscape data was collected by I-PCR amplification of the junctions spanning putative MLV-ERV integration loci using 2 μl of the ligation products, Taq polymerase (Qiagen), and a pair of inverse primers designed from the conserved MLV-ERV sequences. The primer sequences and PCR condition are listed in Table 2. I-PCR amplicons were resolved in a 7.5% polyacrylamide gel for visualization.

Polymorphism Analysis of Genomic TREome (Mouse Mammary Tumor Virus-Type Endogenous Retrovirus [MMTV-ERV]) Superantigen (SAg) Genes

The MMTV-ERV SAg coding sequences were PCR amplified from the genomic DNA (46 of 57 mouse strains) obtained from the Jackson Laboratory using a set of primers (Table 2). Following cloning of the SAg amplicons using a pGEM-T Easy kit from Promega, plasmid DNAs were prepared for 12 colonies picked from each strain using a QIAprep spin miniprep kit and sequenced (Molecular Cloning Laboratories). Eleven mouse strains had no visible SAg coding sequences amplified (C57L/J, CASA/Rk, CAST/EiJ, CZECHII/EiJ, Mus caroli/EiJ, Mus Pahari/Ei, PANCEVO/Ei, PERA/EiJ, PERC/Ei, SKIVE/Ei, and TIRANO/Ei). Within each mouse strain, following identification of a set of unique MMTV-LTR sequences by multiple alignment analyses using Vector NTI (Invitrogen), MMTV-ERVs' SAg open reading frames were examined and translated in silico. Polymorphisms in the putative SAg proteins were visualized using a function in the Excel program (Microsoft).

Results

Diversity in TREome (MLV-ERV) Profiles Among 12 Laboratory Mouse Strains

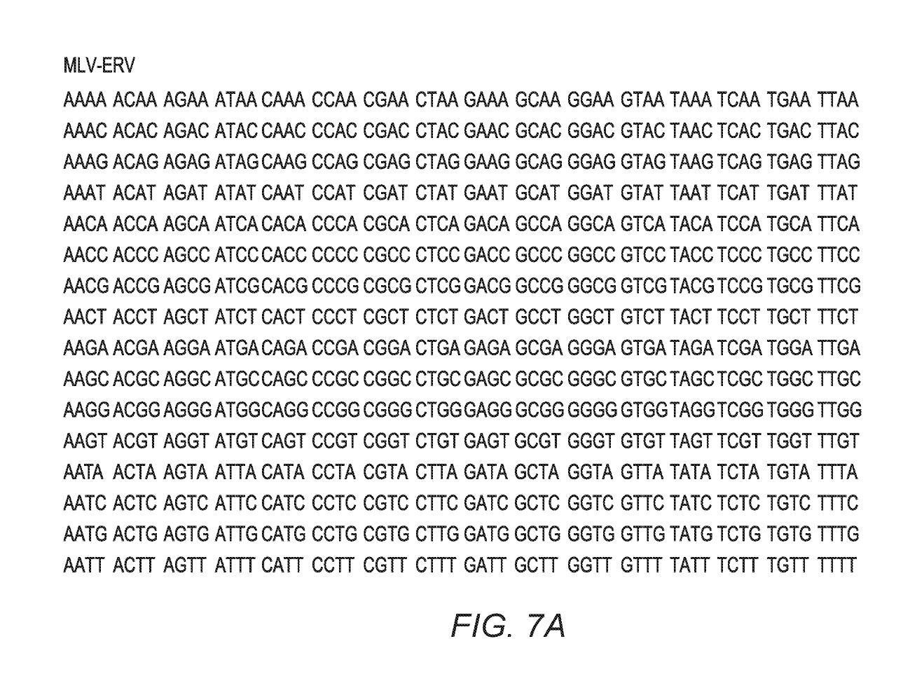

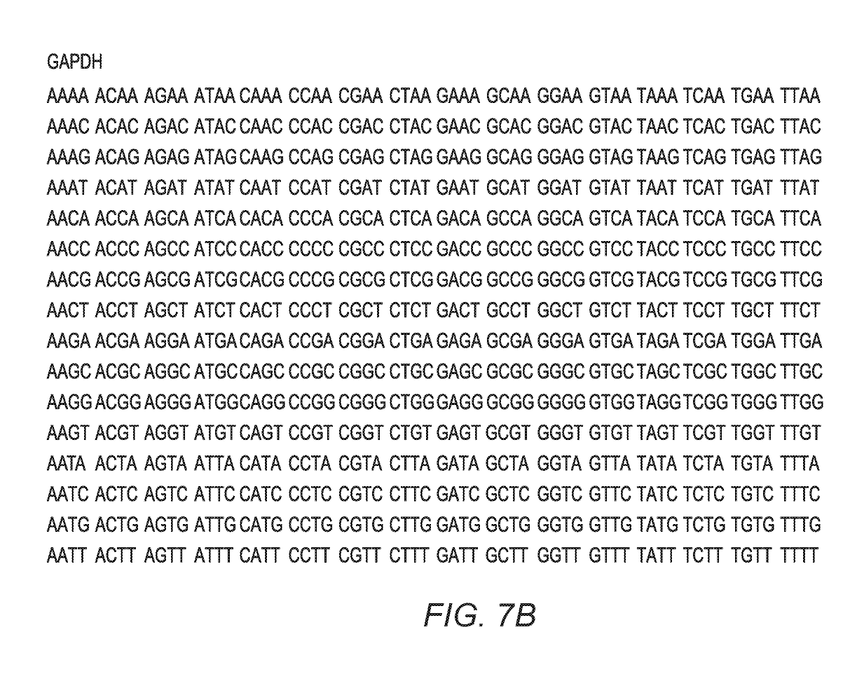

Similarity among the genomes of a range of laboratory mouse strains has been examined primarily based on SNP polymorphism ((Frazer et al., 2007; Kirby et al., 2010; Mekada et al., 2009)). However, it needs to be noted that the genome similarity data was derived mostly from the sequences of conventional genes. To evaluate the extent of TREome diversity among different laboratory mouse strains in comparison to conventional genes, genomic profiles of MLV-ERVs, a mouse TREome family, were examined in 12 laboratory mouse strains (129P1/ReJ, 129X1/SvJ, A/HeJ, A/J, AKR/J, ALR/Lt, BALB/cJ, BDP/J, BPH/2J, BUB/BnJ, C3H/HeJ, and C3H/HeOuJ). The MLV-ERV LTR sequences were isolated from the genomic DNA of each strain and subjected to a probability distribution function analysis for the entire set of all possible four-nucleotide words in order to compute and visualize the variation levels of individual words on a 16×16 (=256) matrix. In contrast to the overall low variation matrix of GAPDH genes derived from 20 laboratory mouse strains including one reference, there were relatively high variations in the vast majority of the words from the MLV-ERV LTR sequences from the 12 inbred mouse strains (FIGS. 7A-B). Interestingly, about a dozen words were highly conserved among the pool of MLV-ERV LTR sequences from 12 mouse strains. Although the genome-wide survey for MLV-ERVs in this study was not comprehensive in nature, the findings from this study provide some evidence that the laboratory mouse population is highly diverse with regard to TREome/MLV-ERV profiles in their genomes in contrast to the relatively high similarity among conventional gene sequences (Frazer et al., 2007; Kirby et al., 2010). Presumably, there would be significant differences in TREome/MLV-ERV profiles between males and females since at least reference mouse chromosome Y is densely populated with both characterized and uncharacterized REs.

Polymorphic TREome Landscapes Among 56 Laboratory Mouse Strains

To further study genomic diversity with regard to TREome profiles among laboratory mouse strains, TREome landscapes were visualized from the genomic DNA of 56 laboratory mouse strains using MLV-ERVs as a probe. At first glance, none of the 56 strains share the same TREome landscape patterns (FIG. 8). However, it was apparent that distinct TREome landscape patterns were shared within certain individual families of strains, such as 129PI-XI/, A/, BALB/, C3H/, C57BL/, DBA/, and MOL, which presumably reflect their common TREome/MLV-ERV ancestries. Interestingly, the TREome landscapes of the C3H/HeJ and its toll-like receptor 4 (TLR4) wildtype control strain (C3H/HeOuJ) were different from each other (jaxmice.jax.org/jaxnotes/archive/430e.html; www.informaticsjax.org/reference/allele/MGI:2386635). In addition, the C57BLKS/J strain's TREome landscape pattern was substantially different from the four other C57BL strains examined. We confirmed that these two sets of strains were the same gender (data not shown), excluding the possibility of chromosome Y effects on the TREome landscape patterns. Furthermore, the TREome landscape patterns of three strains (Mus caroli/Ei, Mus Pahari/Ei, and PANCEVO/Ei) had only a couple of visible amplicon bands while the other 53 strains displayed numerous bands. This finding may coincide with previous reports in which these three strains are placed upstream of the evolutionary pathway of mice compared to the other strains examined in this study (Boursot et al., 1993; Ideraabdullah et al., 2004; Suzuki et al., 2004).

Dissimilar TREome Landscapes Between the C3H/HeJ Strain and its TLR4 Wildtype Control, C3H/HeOuJ

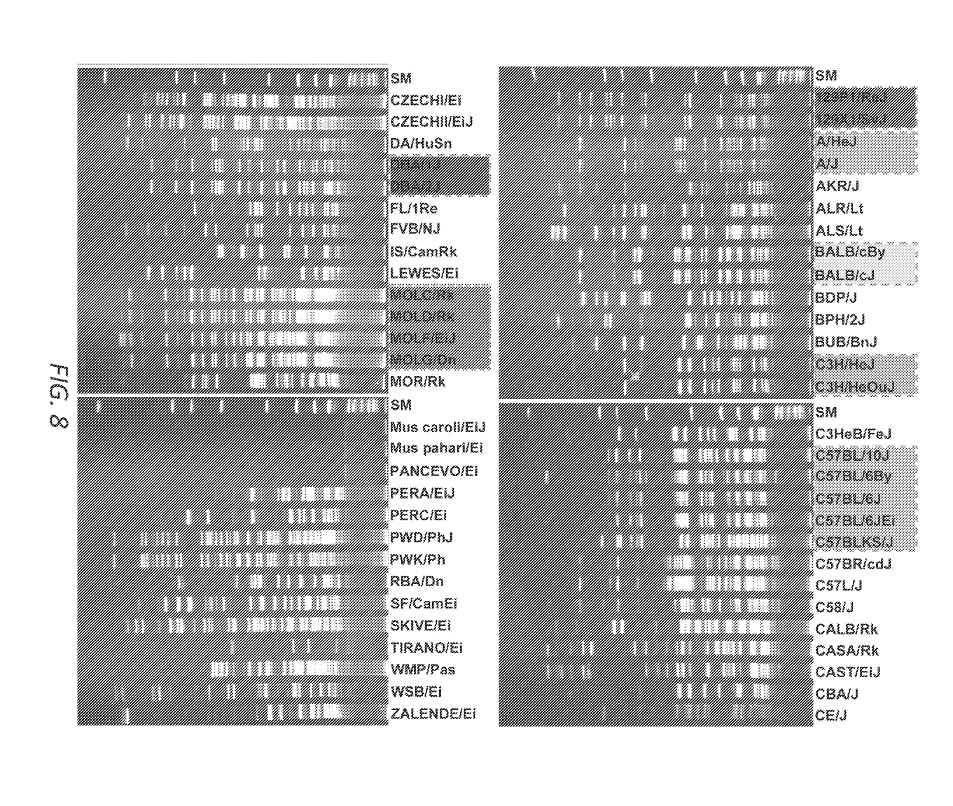

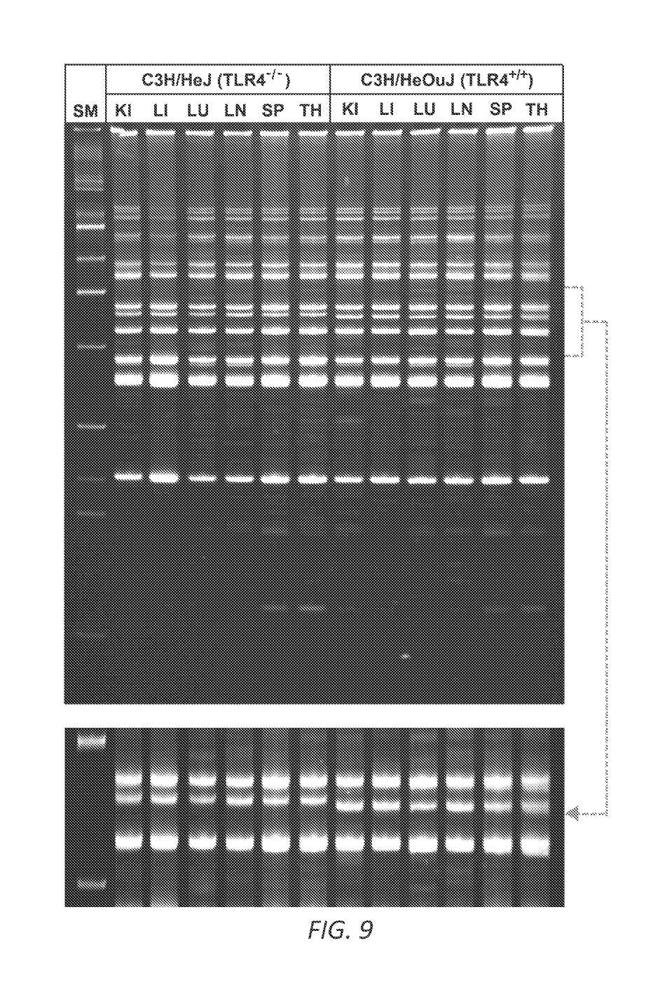

To confirm the initial finding (FIG. 8) that the C3H/HeJ strain (TLR4^{-/-}) is different from its wildtype control strain, C3H/HeOuJ (TLR4^{+/+}), with respect to TREome landscape patterns, we repeated the experiment using six different organs of two additional mice (12-week old females) from each strain. The pair of C3H/HeJ and C3H/HeOuJ strains has been employed widely for the studies focusing on the roles of the TLR4 gene in innate immune functions (Beutler, 2000; Beutler et al., 2001; Poltorak et al., 1998; Smirnova et al., 2000). From all six organs of both strains examined, one distinct TREome band was found only in C3H/HeJ mice whereas another band was only present in C3H/HeOuJ mice (FIG. 9). This difference in banding pattern is consistent with the initial finding described earlier (FIG. 8). In addition, within each mouse, regardless of the strain, there were substantial variations in TREome landscapes among the different organs. The findings from this experiment confirm that in addition to the difference in the TLR4 locus, the genomes of the C3H/HeJ and C3H/HeOuJ strains are different in their TREome landscapes. It is reasonable to assume that application of additional landscaping probes would reveal more TREome loci which are uniquely embedded in the genomes of either C3H/HeJ or C3H/HeOuJ strain.

Un-Identical TREome Landscapes Between the CD14 Knock-Out Strain (CD14^{-/-}) and its Backcross-Control, C57BL/6J (CD14^{+/+}) Strain

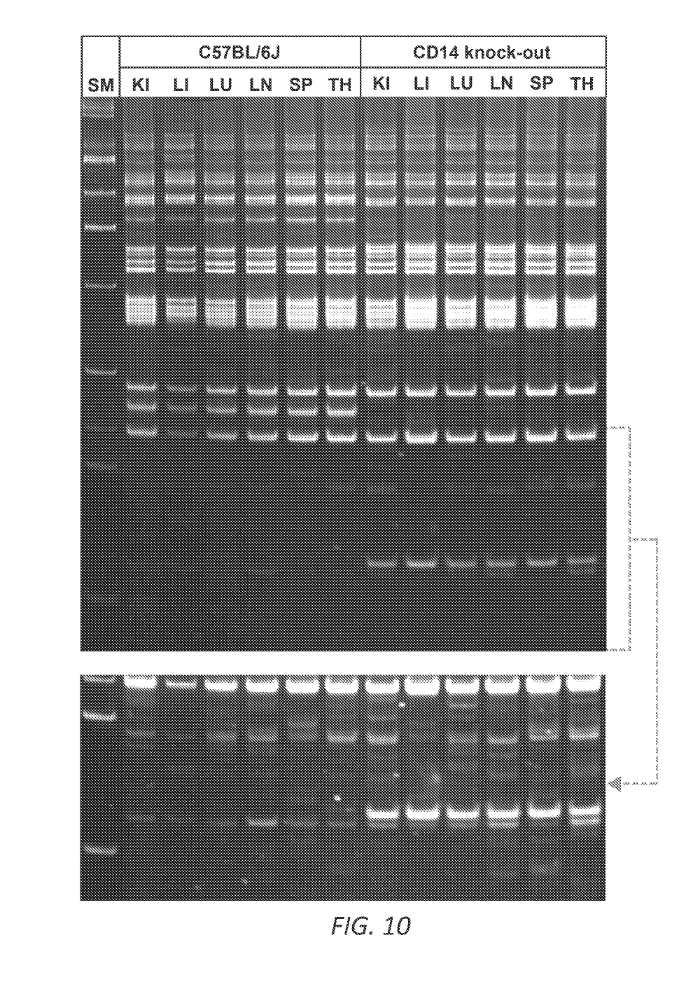

Genetically engineered mouse strains (transgenic or knock-out for a specific target gene) have served as critical and popular components of modern biomedical research efforts (Fox et al., 2006; Houdebine, 2007; Pearson et al., 2008). Typically, the inbred mouse strain, which is introduced during the backcrossing process of generating a genetically engineered strain, is chosen to control the modified/mutated target gene (Seong et al., 2004). In this study, we examined whether there are distinct variations in TREome landscapes between the genomes of a pair of CD14 knock-out (12-week old female) and its backcross-control (C57BL/6J; 12-week old female) strains (Haziot et al., 1996; Poltorak et al., 1998). Within the genomes of all six organs from each strain, two unique TREome/MLV-ERV amplicon bands were visible only in the CD14 knock-out strain, while two other TREome/MLV-ERV amplicon bands were found only in C57BL/6J backcross-control strain (FIG. 10). Similar to the findings from the C3H/HeJ-C3H/HeOuJ pair, the TREome landscapes were variable depending on organs within each strain. The findings from this study indicate that the TREome landscapes of the CD14 knock-out strain are visibly discernable from its backcross-control C57BL/6J strain, in addition to the difference at the genetically targeted locus and its flanking region on chromosome 18 (Cho et al., 2008a; Yee et al., 2008).

Variations in TREome Landscapes in 129 Mouse Substrains

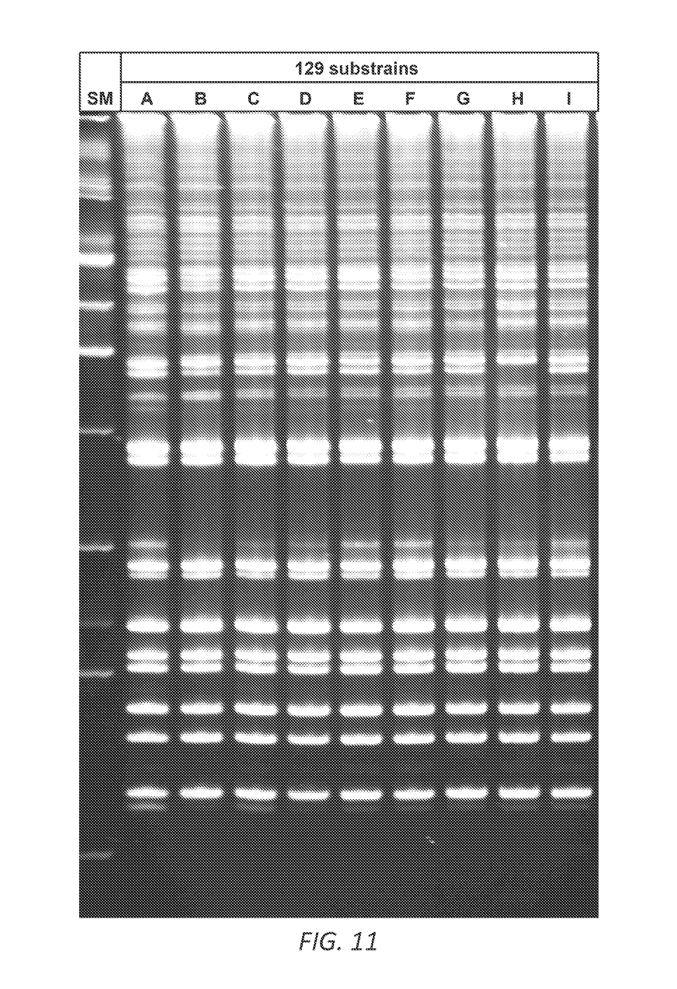

For genetic engineering of transgenic and knock-out mouse models, such as the CD14 knock-out strain described above, embryos of various 129 mouse substrains have been extensively used as a target for the initial manipulation of the genome (Threadgill et al., 1997). Substantial levels of genetic variations among the 129 mouse substrains were reported to be linked to either accidental or intentional outcrossing(s) (Simpson et al., 1997). In this study, genomic DNA from nine 129 substrains (129S1/SvImJ, 129/Sv-Lyn^{tm1Sor}/J 129S1/Sv-Oca2⁺ Tyr⁺ Kitl^{Sl-J}/J, 129S4/SvJae-Inhbb^{tm1Jae}/J, 12954/SvJae-Ppara^{tm1Gonz}/J, 129S4/SvJaeSor-Gt(ROSA)26Sor^{tm1(FLP1)Dym}/J, 129S6/SvEv-Mos^{tm1Ev}/J, 129P1/ReJ, and 129X1/SvJ) were examined to survey variations in the TREome landscapes using an MLV-ERV probe. Overall, all nine 129 substrains shared a common TREome landscape pattern (FIG. 11). However, both of the 129/Sv-Lyn^{tm1Sor}/J and the 129P1/ReJ substrains lacked a unique band in its TREome landscape which is present in the other seven strains. It is interesting to note that the 129S1/SvImJ substrain has a distinct MLV-ERV amplicon band in comparison to the 129/Sv-Lyn^{tm1Sor}/J substrain, for which it often serves as a control. A comprehensive survey of the entire collection of 129 substrains, which employs additional landscaping probes allowing for high-resolution identification of substrain-specific TREome banding patterns, would provide valuable information for phenotypic interrogation of 129-derived genetically engineered strains.

TREome Landscape-Based Surveillance of Genome-Crossing Between Two Mouse Strains

It has been a common practice to backcross chimera mice, which were derived from genetically targeted embryonic genomes of a 129 substrain, with the C57BL/6J strain to establish a stable strain (Hedrich, 2004; Threadgill et al., 1997). In this study, we examined whether the genome-crossing events between two mouse strains (C57BL/6J×129S1/Sv1mJ) are reflected in the TREome landscapes of the hybrid offspring. With regard to the TREome landscape, an F2 hybrid mouse (male), which was derived from an initial crossing of C57BL/6J (female) and 129S1/Sv1mJ (male), was compared to a C57BL/6J mouse (male) and a 129S1/Sv1mJ mouse (male). Although for the most part, the pattern of the F2 hybrid TREome landscape displayed the bands from both C57BL/6J and 129S1/Sv1mJ genomes, it lacked certain bands which were specific only for the individual parental strains (FIG. 12). This incompletely merged hybrid banding pattern within the TREome landscape of the F2 hybrid provides insightful visual information which reveals the static status of genetic crossing between two mouse strains. The F2 hybrid's TREome landscape may be closely linked to the chromosomal cross-over events. High-resolution details of the TREome landscapes can be accomplished by implementing an optimal set of probes derived from MLV-ERVs as well as MMTV-ERVs, LINES, and other TREs/REs. The findings from this study introduce a novel idea that by using the high-resolution TREome landscaping technology, during the courses of backcrossing and/or crossing of mouse strains, the genomes of offsprings at a series of successive generations could be monitored for their “crossing configuration” and uniformity.

Polymorphisms in a “TREome Gene” (MMTV-ERV SAg) Among Laboratory Mouse Strains

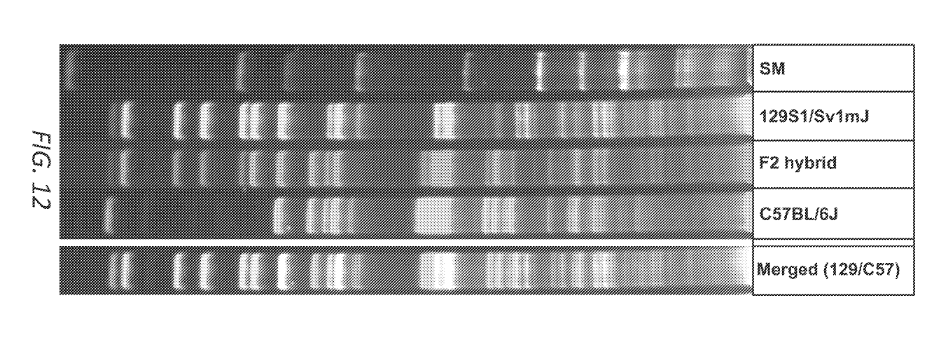

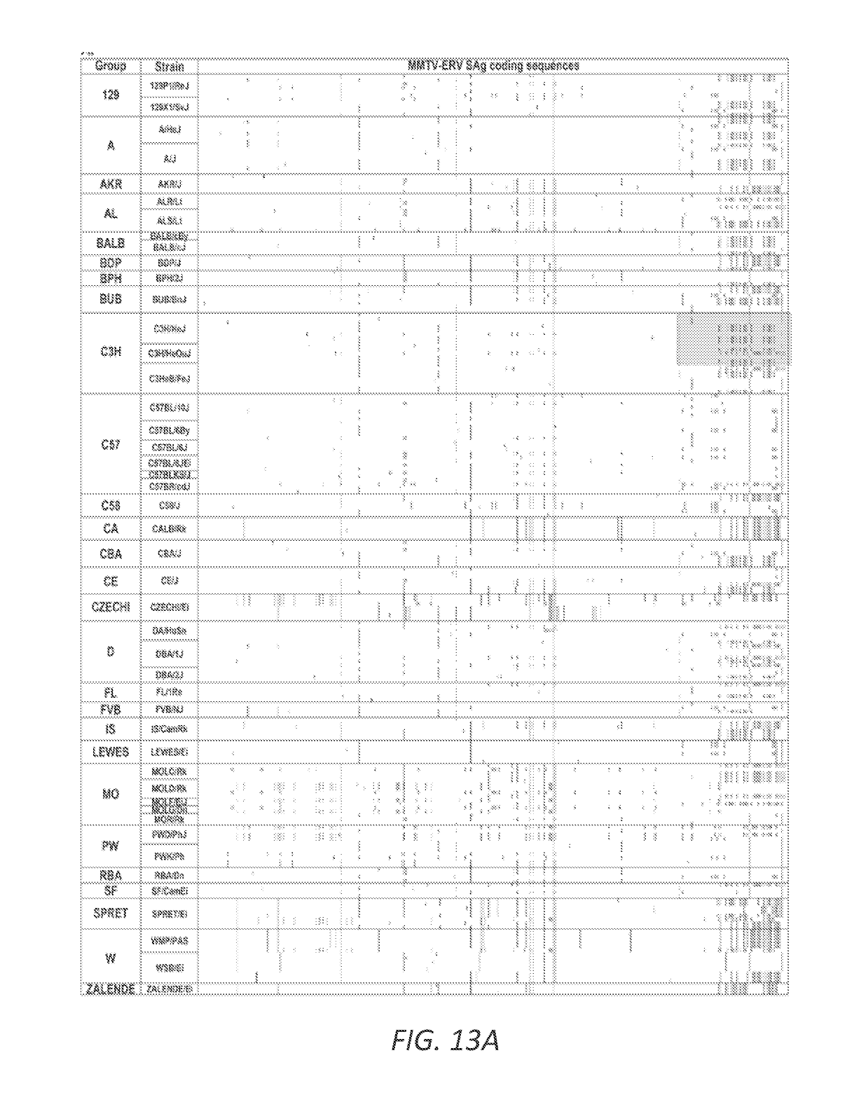

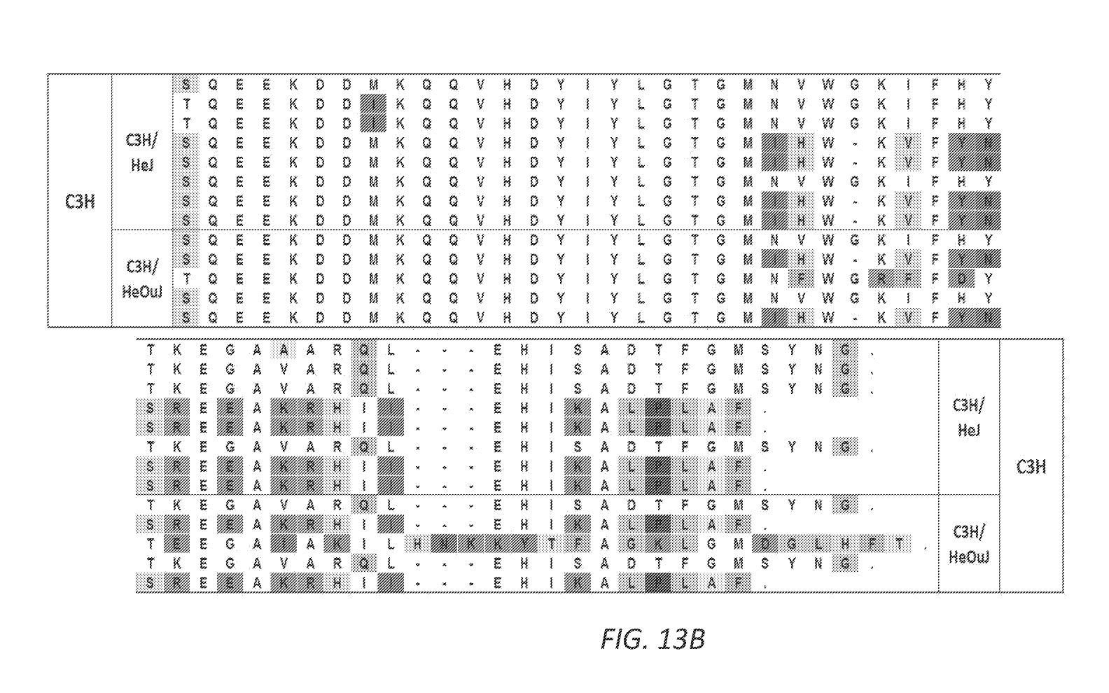

Much of the focus of recent advancements in the genomics and bioinformatics fields hinges on the notion that sequence polymorphisms, including small RNAs, and relevant functions of conventional genes are responsible for phenotypic variations in both normal and disease biology (Mardis, 2008; Mu and Zhang, 2012). To evaluate whether polymorphisms in “TREome genes” cast potential impacts on variable phenotypes among the mouse population, we examined sequence diversity in MMTV-ERV SAg genes, a well-studied immune-regulatory TREome gene (Lee et al., 2011; Peters et al., 1983), among 57 laboratory mouse strains. Only 46 of the 57 mouse strains yielded visible MMTV-ERV LTR amplicons which are presumed to harbor the SAg gene open reading frame (ORF). A high-level of SAg gene polymorphism was indicated by the finding that at least one (up to eight) unique SAg coding sequence was identified within the genomes of the 46 individual mouse strains (FIG. 13A). Altogether, 183 isoforms of the MMTV-ERV SAg gene were derived from the 46 mouse strains. As expected, the C-terminus regions of the SAg ORFs, which are reported to confer the Vβ-specificity of B lymphocytes during interactions with T lymphocytes (Acha-Orbea and MacDonald, 1995), had the highest variations in conjunction with a few polymorphic clusters in the other regions. Interestingly, the genomic profiles of the MMTV-ERV SAg ORFs were different between the C3H/HeJ inbred strain and its TLR4 wildtype control strain, C3H/HeOuJ; Eight and five unique putative SAg isoforms were identified in the genomes of C3H/HeJ and C3H/HeOuJ strains, respectively (FIG. 13B). A well-defined and comprehensive survey would be necessary to confirm the extent of temporal and spatial variations in the MMTV-ERV SAg ORFS which exist in the genomes of these two strains. Polymorphisms in MMTV-ERV SAg, a TREome gene, may differentially shape the immune profiles (e.g., negatively selected T lymphocytes) of the C3H/HeJ and C3H/HeOuJ as a well as other laboratory mouse strains (Tomonari et al., 1993). It is uncertain how the potentially differential systemic immune profiles between C3H/HeJ and C3H/HeOuJ strains, due to the MMTV-ERV SAg polymorphisms, would be networked with the function of TLR4 with regard to its role in innate immunity.

### Example 3. Variable TREomes of a Breast Cancer Patient

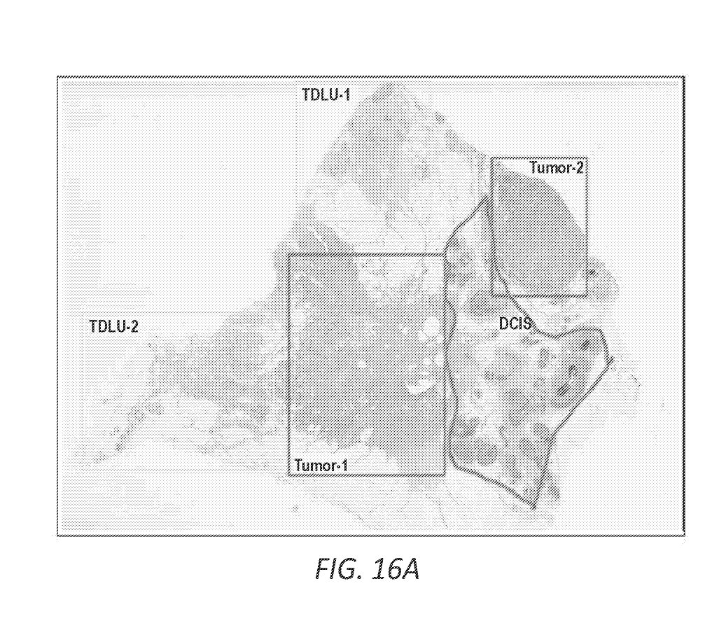

The TREome landscapes of varying areas (normal, precancer, and tumor) in a biopsy sample from a subject diagnosed with breast cancer was evaluated using I-PCR as described above (FIGS. 16A-B), using primers to HERV-K2 and HERV-W as shown in Table 3. Genomic DNAs were isolated from micro-dissected paraffin sections and digested with restriction enzymes before HERV-genomic junctions were amplified by I-PCR. The HERV-junction amplicons were resolved by polyacrylamide gel electrophoresis for TREome landscape evaluation. Although all the genomic DNAs were derived from a single patient, TREome landscape patterns were highly variable depending on the regions of the biopsy sample.

TABLE 3
PCR primers and reaction conditions.

| | | PCR conditions | | | |
|---|---|---|---|---|---|
| Primer | Sequence | Denaturation | Annealing | Elongation | Cycles |
| HERV-K(HML2)(F) | 5′-GTCATCACCAC | 95° C. / | 55° C. / | 72° C. / | 35 |
| | TCCCTAATCT-3′ | 30sec | 60sec | 120sec | |
| HERV-K(HML2)(R) | 5′-GGAAAAGAAA | | | | |
| | AAGACACAGAG-3′ | | | | |
| HERV-W (F) | 5′-AGAGCACAGC | | | | |
| | AGGAGGGA-3′ | | | | |
| HERV-W (R) | 5′-GGGGTCCTTG | | | | |
| | CTCACAGA-3′ | | | | |

As shown in FIGS. 16A-B, the HERV amplicons varied from area to area.

### Example 4. Variable TREomes after Burn Injury in Blood Cells

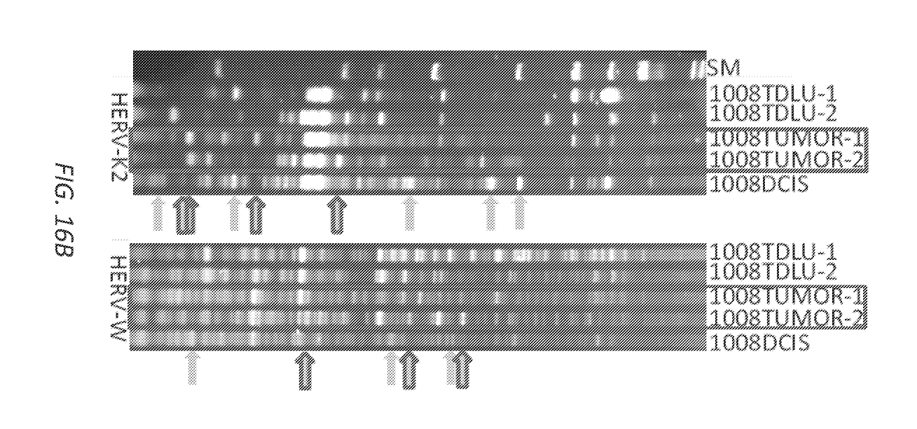

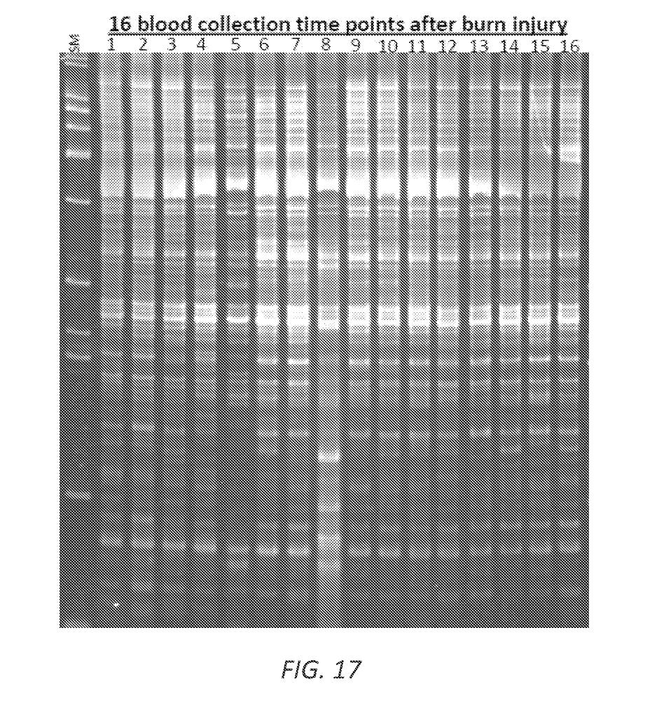

The methods described above were used to evaluate the effects of injury stress on TREome landscape using HERV sequences as probes in a series of blood samples from a subject after a burn injury. As shown in FIG. 17, the TREome landscapes, which are represented by HERV-genomic junction amplicons, showed dynamic alterations in the TREome of the patient's white blood cells after burn injury. In addition, see Lee et al., Experimental and Molecular Pathology 96 (2014) 178-187, in which the data regarding injury-dependent alterations in the expression of TREome/HERVs were discussed.

### Example 5. TREome-Mediated Organ- and Age-Specific Global Changes in Genome Structure in C57BL/6 Inbred Mice

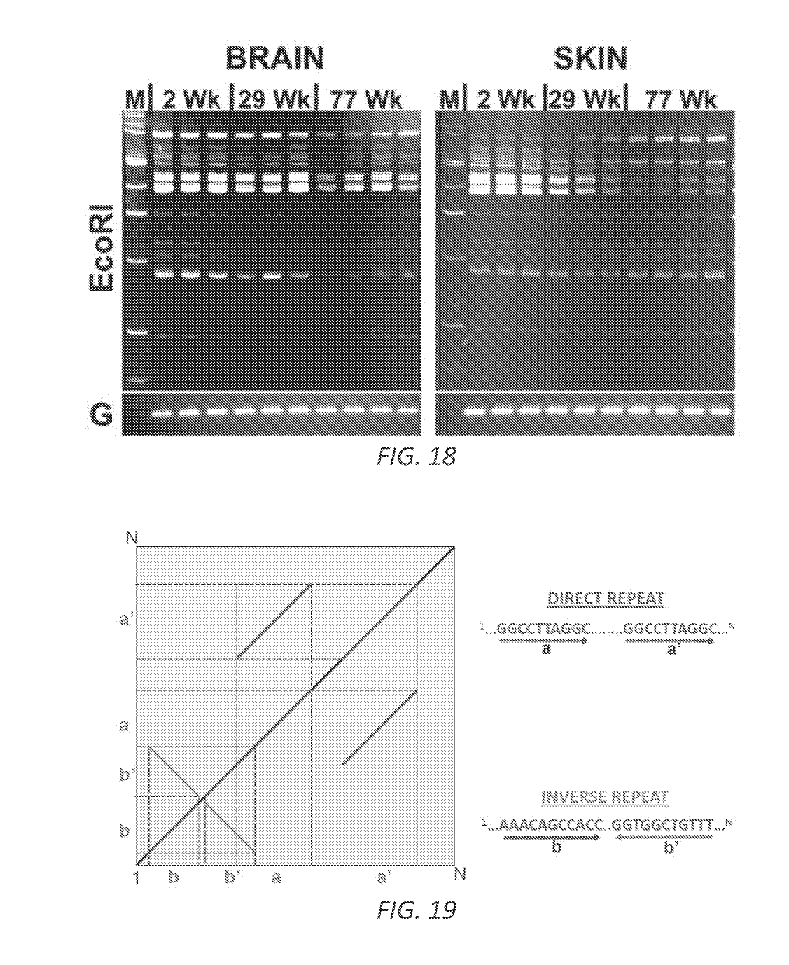

The methods described above were used to analyse the TREome in skin and brain of C57BL/6 inbred mice as they aged. The results, shown in FIG. 18, showed organ- and age-specific global changes in genome structure.

### Example 6. RE Arrays: Species Specific and Ordered Genome Units

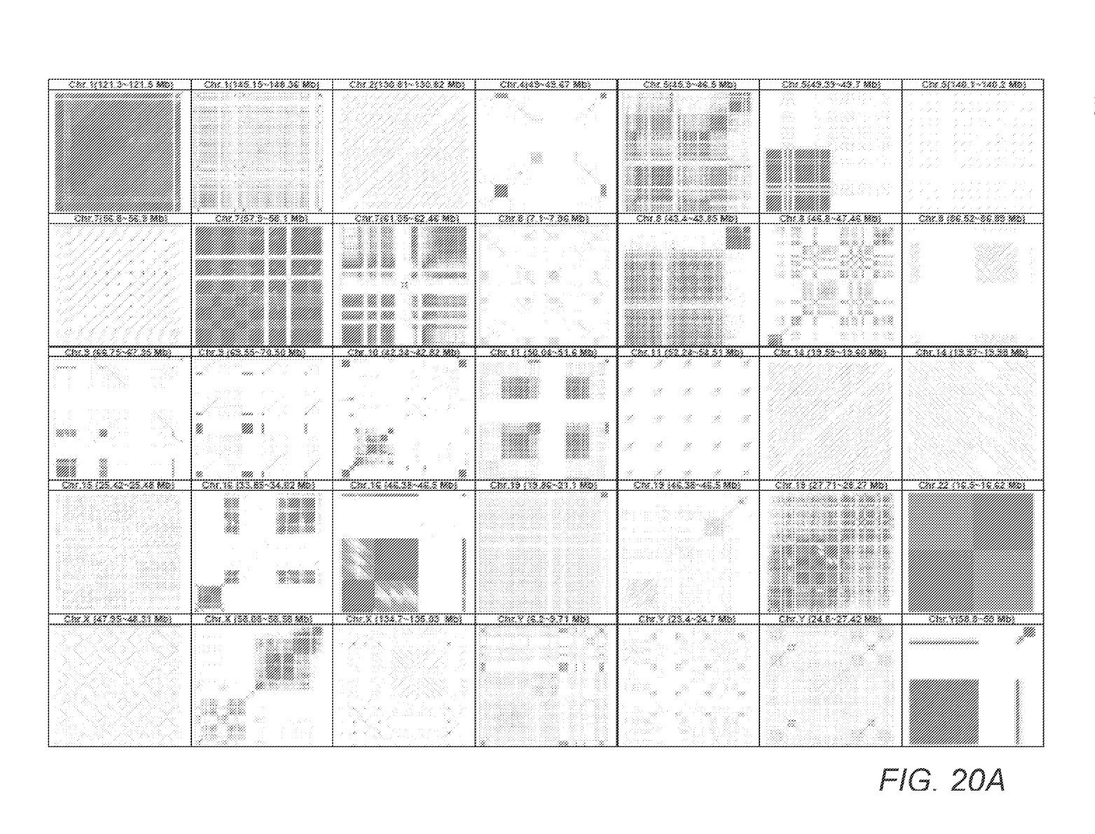

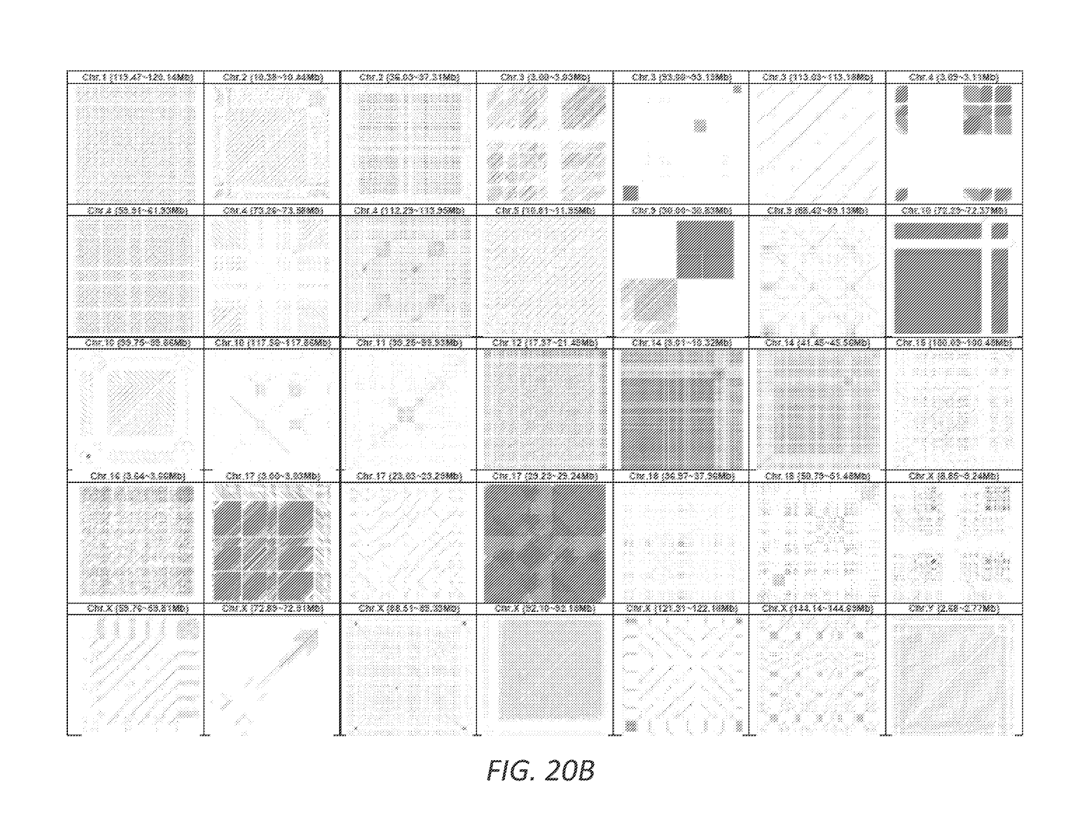

FIGS. 20A and B are sets of exemplary RE arrays from human (A) and mouse (B) showing that the patterns are unique for each species, in contrast to ˜85% sequence homology for conventional genes. These findings suggest that RE arrays contribute to phenotypic details unique for each species.

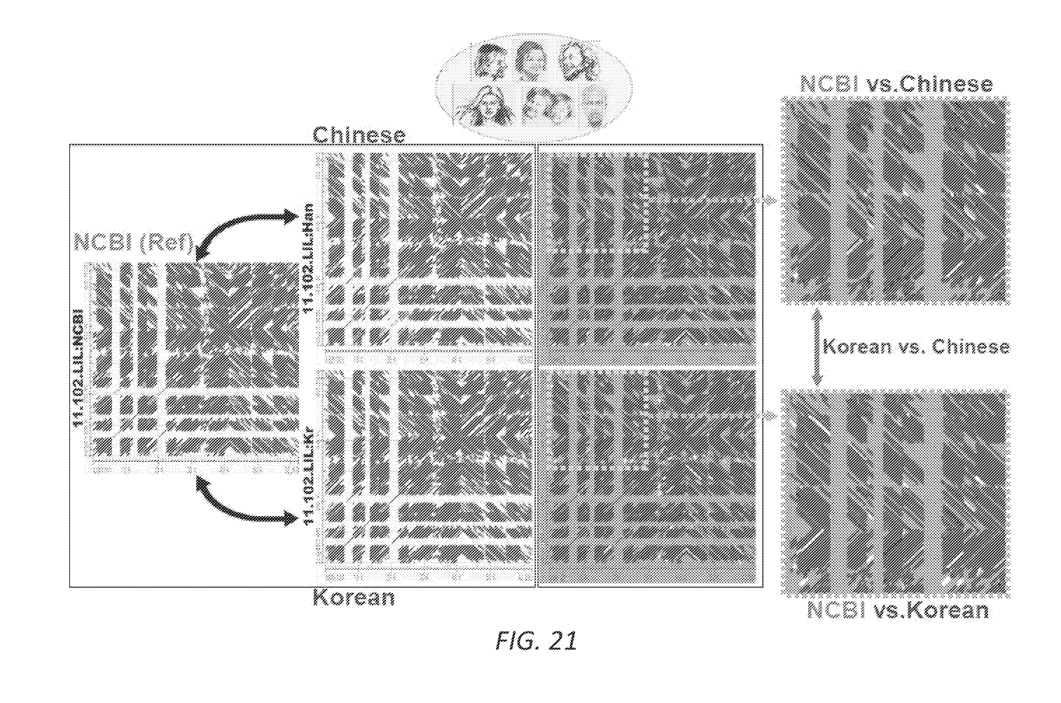

FIG. 21 shows exemplary comparison between the NCBI reference RE array with locus-matching arrays from individuals of Chinese or Korean ancestry. Although the overall configuration of the locus-matching three arrays are almost identical, a close examination identifies multiple configurational polymorphisms unique for the individual arrays of different ancestry.

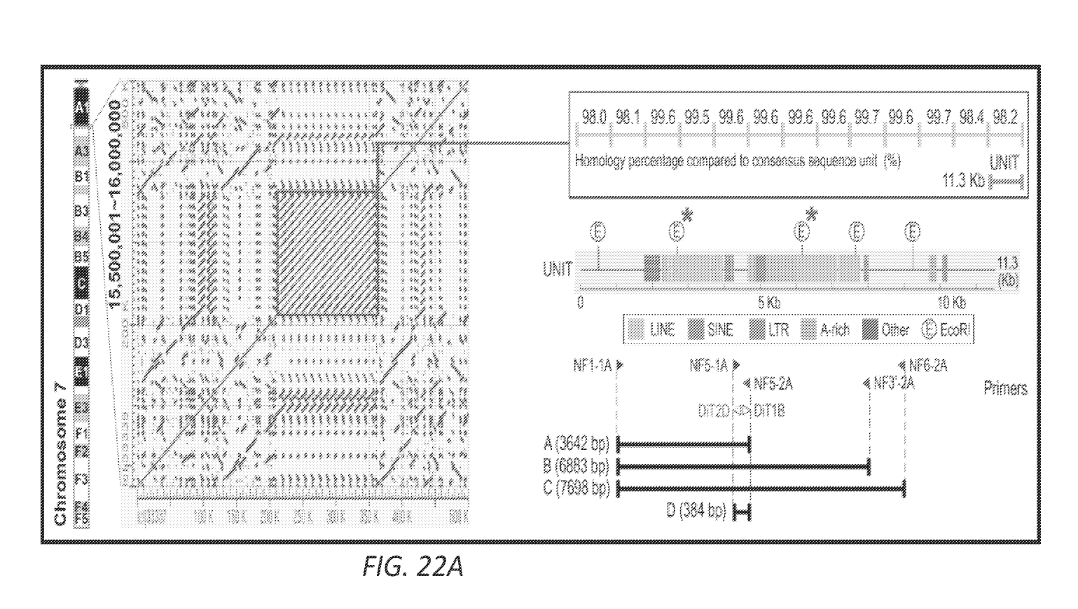

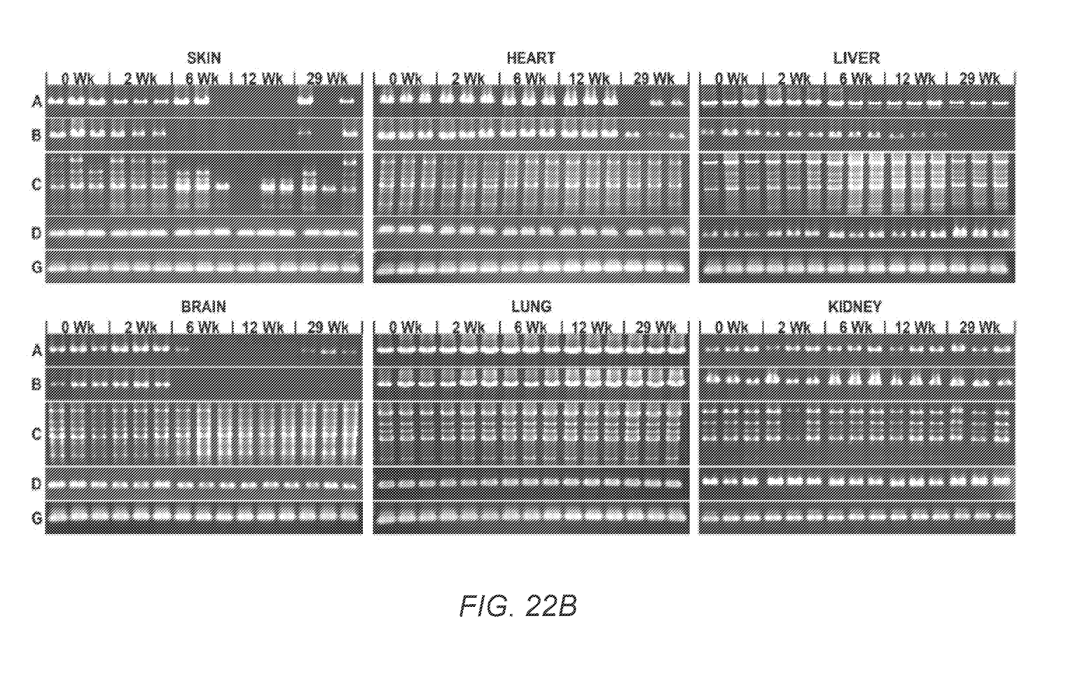

FIG. 22A shows three different PCR strategies for structural analyses of the central tandem repeat cluster within the RE arrayCHR7.32 locus on chromosome 7. The tandem repeat cluster (in both forms of dot-matrix structure and linear-visualization of mosaic repeat units) within the RE arrayCHR7.32 locus is outlined with red lines. PCR primers (Table 4) for three different types of structural analyses of the highlighted tandem repeat cluster are mapped: 1) staggered PCR (A/B/C/D amplicons: NF1-1A, NF5-1A, NF3-2A, NF5-2A, and NF6-2A), 2) repeat unit-length PCR (UL amplicon: Obox4-1B and NF8r-2B), and 3) I-PCR (IN amplicon: DiT-1B and DiT-2D). In addition, putative PCR amplicon sizes expected from the individual primer sets without any recombination events are provided as a reference.

FIG. 22B shows structural variations in the central tandem repeat cluster within the RE arrayCHR7.32 locus. Six organs/tissues from five age groups of C57BL/6J female mice were surveyed for structural variations in the tandem repeat cluster within the RE arrayCHR7.32 locus using staggered PCR primer sets (Table 4). Organ/tissue-specific and age-dependent structural variations in the tandem repeat cluster within the RE arrayCHR7.32 locus were identified in the five age groups (0 week [neonate], 2 weeks, 6 weeks, 12 weeks, and 29 weeks) by examining four sets of the PCR amplicons (A, B, C, and D) which are generated using staggered primer pairs (FIG. 22A). wk (week); G (GAPDH).

TABLE 4
PCR primers and reaction conditions.

| | | | PCR conditions | | | |
|---|---|---|---|---|---|---|
| Target | Primer | Sequence | Denaturation | Annealing | Elongation | Cycles |
| Inverse | DiT1B | 5′-TGACTCAATTAA | 94° C. / 30sec | 53° C. / | 72° C. / | 30 |
| PCR | | TTTTAGTAGC-3′ | | 60sec | 240sec | |
| | DiT2D | 5′-ACAGTTTGACC | | | | |
| | | AGGGGAAGT-3′ | | | | |
| Staggered | NF1-1A | 5′-GCTCTGCCTATC | | | | |
| PCR | | AGGATACCT-3′ | | | | |
| | NF5-1A | 5′-TGGGGGACTCT | | | | |
| | | TGTAAAGTTGC-3′ | | | | |
| | NF3-2A | 5′-GCTACTAAAA | | | | |
| | | TTAATTGAGTC-3 | | | | |
| | NF5-2A | 5′-CCTCAAAATTAT | | | | |
| | | AAATATGCATC-3′ | | | | |
| | NF6-2A | 5′-GGCTGGTACA | | | | |
| | | CAAAAGCACA-3′ | | | | |
| Repeat | Ob0x4- | 5′-CAGACCTGGAA | | | | |
| unit- | 1B | CAACGGAA-3′ | | | | |
| length | NF8R- | 5′-CACCATATTGG | | | | |
| PCR | 2A | ACTCATTCT-3′ | | | | |
| GAPDH | GAPDH | 5′-TGACCACAGTC | 95° C./ 30 sec | 55° C./ 60 | 72° C. / | 25 |
| | rt-1 For | CATGCCATC-3′ | | sec | 60sec | |
| | GAPDH | 5′-GACGGACACAT | | | | |
| | rt-1 Rev | TGGGGGTAG-3′ | | | | |

### REFERENCES

Acha-Orbea, H., MacDonald, H. R., 1995. Superantigens of mouse mammary tumor virus. Annu Rev Immunol. 13, 459-86.
Amid, C., et al., 2009. Manual annotation and analysis of the defensin gene cluster in the C57BL/6J mouse reference genome. BMC Genomics. 10, 606.
Antony, J. M., et al., 2011. Human endogenous retroviruses and multiple sclerosis: innocent bystanders or disease determinants? Biochim Biophys Acta. 1812, 162-76.
Austin, C. P., et al., 2004. The knockout mouse project. Nat Genet. 36, 921-4.
Batzer, M. A., et al., 1996. Genetic variation of recent Alu insertions in human populations. J Mol Evol. 42, 22-9.
Beck, J. A., et al., 2000. Genealogies of mouse inbred strains. Nat Genet. 24, 23-5.
Bennett, E. A., et al., 2004. Natural genetic variation caused by transposable elements in humans. Genetics. 168, 933-51.
Bentvelzen, P., 1992. Immunosuppression by the MMTV superantigen? Immunol Today. 13, 77.
Beutler, B., 2000. Tlr4: central component of the sole mammalian LPS sensor. Current opinion in immunology. 12, 20-26.
Beutler, B., et al., 2001. Identification of Toll-like receptor 4 (Tlr4) as the sole conduit for LPS signal transduction: genetic and evolutionary studies. Journal of endotoxin research. 7, 277-280.
Bittmann, I., et al., 2012. Expression of porcine endogenous retroviruses (PERV) in different organs of a pig. Virology. 433, 329-36.
Bohne, A., et al., 2008. Transposable elements as drivers of genomic and biological diversity in vertebrates. Chromosome Res. 16, 203-15.
Boissinot, S., et al., 2004. The insertional history of an active family of L1 retrotransposons in humans. Genome Res. 14, 1221-31.
Bona, C. A., Revillard, J.-P., 2001. Cytokines and Cytokine Receptors: Physiology and Pathological Disorders. CRC Press.
Boursot, P., et al., 1993. The evolution of house mice. Annual Review of Ecology and Systematics. 119-152.
Cho, K., et al., 2008a. Cosegregation of CD14 locus and polymorphic alleles of glucocorticoid receptor and protocadherins into CD14 knockout mouse genome. Shock. 29, 724-32.
Cho, K., et al., 2008b. Endogenous retroviruses in systemic response to stress signals. Shock. 30, 105-16.
Church, D. M., et al., 2015. Extending reference assembly models. Genome Biol. 16, 13.
Consortium, I. H. G. S., 2004. Finishing the euchromatic sequence of the human genome.
Nature. 431, 931-45.
Consortium, M. G. S., 2002. Initial sequencing and comparative analysis of the mouse genome. Nature. 420, 520-62.
Davisson, M., 1990. The Jackson Laboratory mouse mutant resource.
Doetschman, T., 2009. Influence of genetic background on genetically engineered mouse phenotypes. Methods Mol Biol. 530, 423-33.
Eisener-Dorman, A. F., et al., 2009. Cautionary insights on knockout mouse studies: the gene or not the gene? Brain Behav Immun. 23, 318-24.
Fox, J. G., et al., 2006. The Mouse in Biomedical Research: Normative biology, husbandry, and models. Academic Press.
Fox, R. R., et al., 1997. Handbook on genetically standardized JAX mice. Jackson Laboratory.
Frazer, K. A., et al., 2007. A sequence-based variation map of 8.27 million SNPs in inbred mouse strains. Nature. 448, 1050-3.
Fujimoto, A., et al., 2010. Whole-genome sequencing and comprehensive variant analysis of a Japanese individual using massively parallel sequencing. Nat Genet. 42, 931-6.
Geisler, S., Coller, J., 2013. RNA in unexpected places: long non-coding RNA functions in diverse cellular contexts. Nat Rev Mol Cell Biol. 14, 699-712.
Giachino, C., et al., 2013. Maintenance of genomic stability in mouse embryonic stem cells: relevance in aging and disease. Int J Mol Sci. 14, 2617-36.
Giardine, B., et al., 2007. PhenCode: connecting ENCODE data with mutations and phenotype. Hum Mutat. 28, 554-62.
Gibb, E. A., et al., 2015. Activation of an endogenous retrovirus-associated long non-coding RNA in human adenocarcinoma. Genome Med. 7, 22.
Green, E. L., 1981. Genetics and probability in animal breeding experiments. Macmillan Publishers Ltd.
Guenet, J. L., 2005. The mouse genome. Genome Res. 15, 1729-40.
Haziot, A., et al., 1996. Resistance to endotoxin shock and reduced dissemination of gram-negative bacteria in CD14-deficient mice. Immunity. 4, 407-14.
Hedrich, H., 2004. The laboratory mouse. Academic Press.
Herrero-Medrano, J. M., et al., 2014. Whole-genome sequence analysis reveals differences in population management and selection of European low-input pig breeds. BMC Genomics. 15, 601.
Holder, B. S., et al., 2012. Syncytin 1 in the human placenta. Placenta. 33, 460-6.
Houdebine, L.-M., Transgenic animal models in biomedical research. Target Discovery and Validation Reviews and Protocols. Springer, 2007, pp. 163-202.
Huber, B. T., et al., 1994. The role of superantigens in the immunobiology of retroviruses. Ciba Found Symp. 187, 132-40; discussion 140-3.
Ideraabdullah, F. Y., et al., 2004. Genetic and haplotype diversity among wild-derived mouse inbred strains. Genome research. 14, 1880-1887.
Jun, J., et al., 2014. Whole genome sequence and analysis of the Marwari horse breed and its genetic origin. BMC Genomics. 15 Suppl 9, S4.
Kamath, A. B., et al., 2003. Toll-like receptor 4-defective C3H/HeJ mice are not more susceptible than other C3H substrains to infection with Mycobacterium tuberculosis. Infect Immun. 71, 4112-8.
Kao, D., et al., 2012. ERE database: a database of genomic maps and biological properties of endogenous retroviral elements in the C57BL/6J mouse genome. Genomics. 100, 157-61.
Kim, J. I., et al., 2009. A highly annotated whole-genome sequence of a Korean individual. Nature. 460, 1011-5.
Kim, S., et al., 2003. Stat1 functions as a cytoplasmic attenuator of Runx2 in the transcriptional program of osteoblast differentiation. Genes & development. 17, 1979-1991.
Kirby, A., et al., 2010. Fine mapping in 94 inbred mouse strains using a high-density haplotype resource. Genetics. 185, 1081-1095.
Kotzin, B. L., et al., 1993. Superantigens and their potential role in human disease. Adv Immunol. 54, 99-166.
Lambert, R., 2007. Breeding strategies for maintaining colonies of laboratory mice. A Jackson Laboratory Resource Manual. The Jackson Laboratory.
Lander, E. S., et al., 2001. Initial sequencing and analysis of the human genome. Nature. 409, 860-921.
Lee, K. H., et al., 2012. Age-dependent and tissue-specific structural changes in the C57BL/6J mouse genome. Exp Mol Pathol. 93, 167-72.
Lee, K. H., et al., 2013. Large interrelated clusters of repetitive elements (REs) and RE arrays predominantly represent reference mouse chromosome Y. Chromosome Res. 21, 15-26.
Lee, K. H., et al., 2014. Divergent and dynamic activity of endogenous retroviruses in burn patients and their inflammatory potential. Exp Mol Pathol. 96, 178-87.
Lee, K. H., et al., 2015. Temporal and spatial rearrangements of a repetitive element array on C57BL/6J mouse genome. Exp Mol Pathol. 98, 439-445.
Lee, Y. K., et al., 2011. Prevalent de novo somatic mutations in superantigen genes of mouse mammary tumor viruses in the genome of C57BL/6J mice and its potential implication in the immune system. BMC Immunol. 12, 5.
Li, D., et al., 2013. Heritable gene targeting in the mouse and rat using a CRISPR-Cas system. Nat Biotechnol. 31, 681-3.
Livak, K. J., Schmittgen, T. D., 2001. Analysis of relative gene expression data using real-time quantitative PCR and the 2(-Delta Delta C(T)) Method. Methods. 25, 402-8.
Mardis, E. R., 2008. The impact of next-generation sequencing technology on genetics. Trends Genet. 24, 133-41.
Maronpot, R. R., 2013. The role of the toxicologic pathologist in the post-genomic era(#). J Toxicol Pathol. 26, 105-10.
Mekada, K., et al., 2009. Genetic differences among C57BL/6 substrains. Experimental Animals. 58, 141-149.
Montagutelli, X., 2000. Effect of the genetic background on the phenotype of mouse mutations. J Am Soc Nephrol. 11 Suppl 16, S101-5.
Mouse Genome Sequencing Consortium, 2002. Initial sequencing and comparative analysis of the mouse genome. Nature. 420, 520-62.
Mu, W., Zhang, W., 2012. Bioinformatic Resources of microRNA Sequences, Gene Targets, and Genetic Variation. Front Genet. 3, 31.
Mullikin, J. C., et al., 2010. Light whole genome sequence for SNP discovery across domestic cat breeds. BMC Genomics. 11, 406.
Nakagawa, K., Harrison, L. C., 1996. The potential roles of endogenous retroviruses in autoimmunity. Immunol Rev. 152, 193-236.
Niu, Y., Liang, S., 2009. Genetic differentiation within the inbred C57BL/6J mouse strain. Journal of Zoology. 278, 42-47.
Padyukov, L., 2013. Between the lines of genetic code: genetic interactions in understanding disease and complex phenotypes. Academic Press.
Parisod, C., et al., 2010. Impact of transposable elements on the organization and function of allopolyploid genomes. New Phytol. 186, 37-45.
Pearson, T., et al., Humanized SCID mouse models for biomedical research. Humanized Mice. Springer, 2008, pp. 25-51.
Peters, G., et al., 1983. Tumorigenesis by mouse mammary tumor virus: evidence for a common region for provirus integration in mammary tumors. Cell. 33, 369-77.
Petkov, P. M., et al., 2004. Development of a SNP genotyping panel for genetic monitoring of the laboratory mouse. Genomics. 83, 902-11.
Phelan, J. P., Austad, S. N., 1994. Selecting animal models of human aging: inbred strains often exhibit less biological uniformity than F1 hybrids. Journal of gerontology. 49, B1-B11.
Poltorak, A., et al., 1998. Defective LPS signaling in C3H/HeJ and C57BL/10ScCr mice: mutations in Tlr4 gene. Science. 282, 2085-2088.
Rosenfeld, J. A., et al., 2012. Limitations of the human reference genome for personalized genomics. PLoS One. 7, e40294.
Rossant, J., 2013. Making a knockout mouse: From stem cells to embryos. Nat Cell Biol. 15, 1133.
Schmitt, K., et al., 2015. HERV-K(HML-2) rec and np9 transcripts not restricted to disease but present in many normal human tissues. Mob DNA. 6, 4.
Seok, J., et al., 2013. Genomic responses in mouse models poorly mimic human inflammatory diseases. Proc Natl Acad Sci USA. 110, 3507-12.
Seong, E., et al., 2004. To knockout in 129 or in C57BL/6: that is the question. Trends Genet. 20, 59-62.
Shastry, B. S., 2002. SNP alleles in human disease and evolution. J Hum Genet. 47, 561-6.
Sigmund, C. D., 2000. Viewpoint: are studies in genetically altered mice out of control? Arterioscler Thromb Vasc Biol. 20, 1425-9.
Silver, L. M., 1995. Mouse genetics: concepts and applications. Oxford University Press.
Simpson, E. M., et al., 1997. Genetic variation among 129 substrains and its importance for targeted mutagenesis in mice. Nature genetics. 16, 19-27.
Smirnova, I., et al., 2000. Phylogenetic variation and polymorphism at the toll-like receptor 4 locus (TLR4). Genome Biol. 1, 1-002.10.
Suzuki, H., et al., 2004. Temporal, spatial, and ecological modes of evolution of Eurasian Mus based on mitochondrial and nuclear gene sequences. Mol Phylogenet Evol. 33, 626-46.
Taft, R. A., et al., 2006. Know thy mouse. Trends Genet. 22, 649-53.
Takao, K., Miyakawa, T., 2015a. Correction for Takao and Miyakawa, Genomic responses in mouse models greatly mimic human inflammatory diseases. Proc Natl Acad Sci USA. 112, E1163-7.
Takao, K., Miyakawa, T., 2015b. Genomic responses in mouse models greatly mimic human inflammatory diseases. Proc Natl Acad Sci USA. 112, 1167-72.
Thomas, K. R., Capecchi, M. R., 1987. Site-directed mutagenesis by gene targeting in mouse embryo-derived stem cells. Cell. 51, 503-12.
Threadgill, D. W., et al., 1997. Genealogy of the 129 inbred strains: 129/SvJ is a contaminated inbred strain. Mamm Genome. 8, 390-3.
Tomonari, K., et al., 1993. Influence of viral superantigens on V beta- and V alpha-specific positive and negative selection. Immunol Rev. 131, 131-68.
Venter, J. C., et al., 2001. The sequence of the human genome. Science. 291, 1304-51.
Walsh, C. P., et al., 1998. Transcription of IAP endogenous retroviruses is constrained by cytosine methylation. Nat Genet. 20, 116-7.
Weichman, K., Chaillet, J. R., 1997. Phenotypic variation in a genetically identical population of mice. Mol Cell Biol. 17, 5269-74.
Yee, K. S., et al., 2008. The effect of CAG repeat length polymorphism in the murine glucocorticoid receptor on transactivation potential. Exp Mol Pathol. 84, 200-5.
Zhang, W., et al., 2014. Whole genome sequencing of 35 individuals provides insights into the genetic architecture of Korean population. BMC Bioinformatics. 15 Suppl 11, S6.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of determining a genetic identity of a cell, tissue, organ, or organism, the method comprising:
determining type, position, and size of every occurrence of at least one repetitive element in the genome of the cell, tissue, or organism;
thereby determining the genetic identify of the cell, tissue, organ, or organism.

2. A computer-implemented method of generating a graphical representation of the genetic identity of a cell, tissue, organ, or organism, the method comprising:
optionally determining type, position, and size of every occurrence of at least one repetitive element in the genome of the cell, tissue, or organism;
receiving electronic information regarding the type, position, and size of every occurrence of at least one repetitive element in the genome of the cell, tissue, organ, or organism; and
using a processor to generate a graphical representation of the electronic information.

3. The method of claim 1 or 2, wherein the cell, tissue, organ, or organism is, or is from, an animal, e.g., a mammal, bird, fish, or reptile; plant; fungus; or bacterium.

4. The method of claim 1, wherein the assay comprises using PCR and/or inverse-PCR (I-PCR) to determine position and sequencing to determine type, size, and/or copy number.

5. The method of claim 2, wherein the electronic information was obtained using PCR and/or inverse-PCR (I-PCR) to determine position and sequencing to determine type, size, and/or copy number.

6. The method of claim 1 or 2, wherein the repetitive element is a Transposable Repetitive Element (TRE).

7. The method of claim 1 or 2, wherein the repetitive element is a non-transposable repetitive element.

8. The method of claim 7, wherein the TRE is an endogenous retrovirus (ERV), long interspersed nuclear element (LINE), short interspersed nuclear element (SINE), or DNA transposon.

9. The method of claim 1 or 2, wherein the type is based on primary sequence; the position is relative to a reference genome; and/or the size refers to the length or number of repeats.

10. The method of claim 2, wherein using a processor to generate a graphical representation of the electronic information comprises unbiased self-alignment and dot-matrix plot visualization.

11. The computer-implemented method of claim 2, further comprising displaying the graphical representation electronically on a display device to provide a visible image.

12. The method of claim 1 or 2, wherein the genetic identity is determined at a specific time or space.

13. The method of claim 1 or 2, wherein the genetic identity is determined at a first time or space, and the method further comprising determining genetic identity at a second time or space, and comparing the genetic identity at the first and second time or space to detect changes in the genetic identity of the cell or organism.

14. The method of claim 13, wherein the second time is later than the first time, and/or the second space is obtained from a different cell, tissue, or organ within the same organism.

15. The method of claim 13, wherein the first and second time or space reflects changes in a disease state in the cell, tissue, organ, or organism.

16. The method of claim 15, further comprising identifying one or more risk factors or prognostic factors based on the changes in the disease state.

17. A computer-implemented method for determining genetic identity of a cell, tissue, organ, or organism, comprising:
accessing, by one or more processing devices, a database to obtain data elements comprising genomic sequence information, gene information, genetic variation information, and repetitive element information for a cell, tissue, organ, or organism at a selected time and/or space;
computing a genetic identity for the cell, tissue, organ, or organism at the selected time and/or space, wherein the genetic identity is computed based on the data elements; and
storing, at a storage location, a representation of the genetic identity.

18. A computer-implemented method, comprising:
accessing, by one or more processing devices, a database to obtain data elements comprising genomic sequence information, gene information, genetic variation information, and repetitive element information for a cell, tissue, organ, or organism at a selected time and/or space;
obtaining additional information relating to genomic sequence information, gene information, genetic variation information, and repetitive element information in the cell, tissue, organ, or organism, wherein the additional information is associated with a predetermined time and/or space, e.g., aging, stress, and/or disease; and
updating the data elements.

19. The method of claim 18, further comprising computing a genetic identity for the cell, tissue, organ, or organism, wherein the genetic identity is computed based on the data elements; and
storing, at a storage location, a representation of the genetic identity.

20. A computer-implemented system for storing genomic information, comprising:
memory storing computer-readable instructions,
one or more processing devices configured to execute the computer-readable instructions to perform operations comprising:
accessing a database to obtain data elements comprising genomic sequence information, gene information, genetic variation information, and repetitive element information for a cell, tissue, organ, or organism at a selected time and/or space;
computing a genetic identity for the cell, tissue, organ, or organism at the selected time and/or space, wherein the genetic identity is computed based on the data elements; and
storing, at a storage location, a representation of the genetic identity.

21. The method of claims 17-20, wherein the representation of the genetic identity is usable for generating an image of the genetic identity.

22. The method of claim 21, further comprising presenting the image of the genetic identity on a display device.

23. The method of claims 17-20, wherein the selected time and/or space relates to changes associated with aging, stress, and/or disease.

24. A method of determining origin of a test subject, the method comprising:
determining type, position, and size of every occurrence of at least one repetitive element in the genome of the test subject;
comparing the type, position, and size of every occurrence of the repetitive element of the test subject to the type, position, and size of every occurrence of the repetitive element of a reference subject;
determining that the type, position, and size of every occurrence of the repetitive element of the test subject and the type, position, and size of every occurrence of the repetitive element of the reference subject is not statistically different; and
identifying the test subject as having the same origin as the reference subject.

25. The method of claim 24, wherein the test subject is a human, a plant, or an animal.

26. A method of sub-classifying a disease of humans, plants, and animals, the method comprising:
determining type, position, and size of every occurrence of at least one repetitive element in the genome of a group of subjects with a disease;
applying a clustering algorithm to the type, position, and size of every occurrence of the repetitive element in the genome of the group of subjects; and
identifying a sub-group of subjects as having a sub-group disease.

27. A method of determining whether a test cell belongs to a reference cell line, the method comprising:
determining type, position, and size of every occurrence of at least one repetitive element in the genome of the test cell;
comparing type, position, and size of every occurrence of the repetitive element in the genome of the test cell to type, position, and size of every occurrence of the repetitive element in the genome of a reference cell from the reference cell line;
determining that the type, position, and size of every occurrence of the repetitive element of the test cell is not statistically different from the type, position, and size of every occurrence of the repetitive element of the reference cell; and
identifying the cell as belonging to the cell line.

28. A method of identifying a locus associated with a disease, the method comprising:
determining type, position, and size of every occurrence of at least one repetitive element in the genome of a first sibling with the disease;
comparing type, position, and size of every occurrence of the repetitive element in the genome of the first sibling to type, position, and size of every occurrence of the repetitive element in the genome of a second sibling, wherein the second sibling does not have the disease; and
identifying the locus associated with the disease.

29. The method of claim 28, wherein the first sibling and the second sibling are of the same sex.
